(19) United States
(12) Patent Application Publication
(10) Pub. No.: US 2011/0236927 A1
(43) Pub. Date: Sep. 29, 2011

(54) GENETICALLY STABILIZED TANDEM GENE DUPLICATION

(75) Inventors: Gregory Stephanopoulos, Winchester, MA (US); Keith Edward Jaggard, Evansto, IL (US)

(73) Assignee: MASSACHUSETTS INSTITUTE OF TECHNOLOGY, CAMBRIDGE, MA (US)

(21) Appl. No.: 13/060,822

(22) PCT Filed: Aug. 27, 2009
(86) PCT No.: PCT/US09/04876

§ 371 Date: Jun. 20, 2011

Related U.S. Application Data
(60) Provisional application No. 61/190,292, filed on Aug. 27, 2008.

(51) Int. Cl. C12P 21/00; C12N 15/63; C12P 1/00; C12N 15/70; C12N 15/81

(52) U.S. Cl. 435/69.1; 435/320.1; 435/440; 435/41; 435/471

(57) ABSTRACT

The invention provides constructs and methods for producing genetically stabilized tandem gene duplications.

### RELATED APPLICATIONS

This application claims the benefit under 35 U.S.C. §119(e) of U.S. Provisional Application Ser. No. 61/190,292, entitled “Genetically Stabilized Tandem Gene Duplication,” filed on Aug. 27, 2008, which is herein incorporated by reference in its entirety.

### GOVERNMENT INTEREST

This work was funded in part by the National Science Foundation under grant number CBET-0730238. The government has certain rights in this invention.

### FIELD OF THE INVENTION

The invention provides constructs and methods for producing genetically stabilized tandem gene duplications.

### BACKGROUND OF THE INVENTION

Although plasmids have been used for over three decades for high copy expression, fundamental flaws in plasmid propagation prohibit their usefulness in long term expression. Mutations that result in loss of expression in one copy of a plasmid are quickly propagated to all copies by “allele segregation,” resulting in a loss of productivity. This rapid propagation is driven by (a) the possibility that one daughter cell can inherit both copies of duplicated mutant plasmid and (b) a growth advantage for fewer copies of the non-mutated allele. No plasmid-stability techniques to date have addressed this problem.

Genomic integration methods are cumbersome to integrate several copies. Methods for genomic integration include those described in U.S. Pat. No. 5,861,273, U.S. Pat. No. 5,395,763 and Diederich et al. (Plasmid 28:14-24, 1992). U.S. Pat. No. 5,395,763 describes the use of a Mu phage genome-based system that leads to duplication of sequences of interest by integration of copies of the Mu prophage into the genome of the host cell. This can lead to insertional mutagenesis of host cell genes. Diederich et al. describes a set of multicopy plasmid vectors that provide for integration into the attB site of the E. coli genome. U.S. Pat. No. 5,861,273 sought to overcome the detrimental features of the other genomic integration methods by use of a circular, non-self replicating DNA called a “chromosomal transfer DNA”. In addition to lacking an origin of replication or an autonomously replicating sequence in the chromosomal transfer DNA used to introduce a gene encoding a protein of interest to a host cell, this method requires that the gene encoding the protein of interest is at no time operably linked to a promoter functional in a host cell on a multicopy plasmid vector during construction of the transfer DNA. This latter feature is included to avoid toxic or lethal effects of the expression of the protein of interest in the host cell prior to integration of the chromosomal transfer DNA. U.S. Pat. No. 5,861,273 describes amplification of the inserted sequences by chromosomal duplication facilitated by flanking the sequences of interest and a selectable marker with duplicate DNA sequences followed by selection for the selectable marker, or by replicative transposition. The methods described in U.S. Pat. No. 5,861,273, U.S. Pat. No. 5,395,763 and Diederich et al. thus require cumbersome manipulations and/or produce amplification by a integrative or transposition methods that can be deleterious to the host cell receiving a sequence of interest by genomic integration.

### SUMMARY OF THE INVENTION

Tandem gene duplication (TGD) has been developed to provide stable, tunable, high-level expression of proteins. TGD can amplify genomic integrations to as many as 50 copies, which are completely genetically stabilized upon deletion of recA. These constructs require no selection markers to maintain copy number, and by being physically linked in one strand of DNA, avoid allele segregation, extending genetic stability 10 fold. The polyhydroxybutyrate (PHB) operon was engineered in Escherichia coli using this method, and actively produced PHB beyond 70 generations at levels plasmids could maintain for only 30 generations. Additionally, the lycopene operon was engineered using this method, leading to a significant increase in lycopene yield. TGD is superior to plasmids for high-level, selection marker-free, long-term recombinant expression.

According to one aspect of the invention, methods for producing a genetically stable tandem gene duplication are provided. The methods include integrating into a chromosome of a host cell a nucleic acid construct comprising a nucleic acid sequence that encodes one or more proteins operably linked to one or more promoter sequences, a nucleic acid sequence encoding a selectable marker, and homologous nucleic acid segments flanking the nucleic acid sequences that encode the one or more proteins and the selectable marker, wherein the host cell comprises a functional recombinase, selecting for tandem gene duplication (TGD) of the nucleic acid sequences that encode the one or more proteins and the selectable marker by culturing the host cell under selective conditions in which the selectable marker confers a growth advantage to the host cell, wherein the TGD is mediated by the functional recombinase, and stabilizing the TGD by deleting the recombinase or disabling the recombinase.

In some embodiments, increasing the number of copies of the nucleic acid sequence encoding the selectable marker confers increasing growth advantage to the host cell.

In other embodiments, the homologous nucleic acid segments are at least 50% identical, more preferably at least 60%, 70%, 80%, 90% or 95% identical. More preferably still, the homologous nucleic acid segments are identical.

In some embodiments, the protein is a protein that is non-native to the host cell.

In other embodiments, the recombinase is encoded by the host cell. Preferably the recombinase is encoded by recA.

In some embodiments, the nucleic acid sequence encoding the selectable marker is an antibiotic resistance gene, preferably a chloramphenicol resistance gene or a tetracycline resistance gene. In such embodiments, the selective conditions include culturing the cells in medium that contains the corresponding antibiotic, preferably chloramphenicol or tetracycline.

In other embodiments, the nucleic acid sequence encoding the selectable marker is an auxotrophic marker gene. In such embodiments, the selective conditions include culturing the cells in a medium that does not supply the metabolite produced by the auxotrophic marker gene.

In some embodiments, the step of selecting for TGD includes successive rounds of culture of the cell under culture conditions that successively require an increase in the number of copies of the nucleic acid sequence encoding the selectable marker.

In some embodiments, the host cell is a bacterial cell, preferably an E. coli cell.

In other embodiments, the host cell is a eukaryotic cell, preferably a yeast cell.

In certain embodiments, there are two homologous nucleic acid segments flanking the nucleic acid sequences that encode the protein and the selectable marker. Preferably these flanking homologous nucleic acid segments are at least 25 nucleotides in length. More preferably, the flanking homologous nucleic acid segments are at least 50, 100, 200, 300, 400, 500, 600, 700, 800 or 900 nucleotides in length. More preferably still, the flanking homologous nucleic acid segments are at least 1000 nucleotides in length. In other embodiments, the flanking homologous nucleic acid segments are less than: 50%, 40%, 30%, 20%, 10%, 5%, 4%, 3%, 2%, or 1% identical with the genome of the host cell. In some embodiments, the flanking homologous nucleic acid segments are sufficiently non-identical with the genome of the host cell that they cannot recombine with the genome of the host cell in the presence of the functional recombinase, and are sufficiently homologous with each other that they can recombine with each other in the presence of the functional recombinase.

In some embodiments, the flanking homologous nucleic acid segments are derived from a genome other than that of the host cell. In certain embodiments, the host cell is a bacterial cell and the flanking homologous nucleic acid segments are derived from a genome of a different species of bacteria or a eukaryotic cell genome. In one embodiment, the host cell is an E. coli cell and the flanking homologous nucleic acid segments are derived from a Synechocystis genome. In some embodiments, the flanking homologous nucleic acid segments are non-coding.

In certain embodiments, the nucleic acid sequence that encodes the one or more proteins is inserted in the construct in a multiple cloning site between the flanking homologous nucleic acid segments.

In some embodiments, the cell is not cultured under the selective conditions after the tandem gene duplication is stabilized by deleting the recombinase or disabling the recombinase.

In certain embodiments, the nucleic acid sequence that encodes the one or more proteins is a phaECAB, CrtEBI, or dxs-idi-ispDF operon.

In other embodiments, the one or more promoter sequences is one or more promoters that is/are dependent on the native RNA polymerase of the host cell.

According to another aspect of the invention, cells produced by the method of any of the foregoing methods are provided. Also provided are containers containing a plurality of such cells, and cell cultures of such cells.

According to a further aspect of the invention, methods for producing a protein or metabolite are provided. The methods include culturing a cell produced by any of the foregoing methods in culture medium. In some embodiments, the methods further include isolating and/or purifying the protein or metabolite from the cell or culture medium.

According to another aspect of the invention, nucleic acid constructs are provided. The nucleic acid constructs include a nucleic acid sequence that encodes one or more proteins operably linked to one or more promoter sequences functional in a host cell, a nucleic acid sequence that encodes a selectable marker, and homologous nucleic acid segments flanking the nucleic acid sequence that encodes the one or more proteins and the nucleic acid sequence that encodes the selectable marker. The nucleic acid sequence that encodes the one or more proteins is operably linked to the one or more promoter sequences on a multicopy number plasmid vector having an origin of replication during construction of the construct. In some embodiments, the origin of replication is a bacterial origin of replication.

In certain embodiments, the features of the nucleic acid sequences, proteins, selectable markers, flanking homologous nucleic acid segments are as described above.

According to another aspect of the invention, cells are provided that include the foregoing nucleic acid construct. Also provided are containers containing a plurality of such cells and cell cultures of such cells.

These and other aspects of the invention, as well as various embodiments thereof, will become more apparent in reference to the drawings and detailed description of the invention.

### BRIEF DESCRIPTION OF THE DRAWINGS

FIG. 1 presents a schematic depicting how chemically induced chromosomal evolution (CIChE) can evolve the chromosome of a microorganism to produce many copies of a recombinant allele. FIG. 1A depicts the CIChE DNA cassette containing the gene(s) of interest and a selectable marker (indicated as “B”), flanked by 1-kb homologous regions (indicated as “A”). This cassette is delivered to the chromosome by standard methods. FIG. 1B depicts how iterative tandem gene duplication is accomplished by recA-mediated DNA crossover between the leading homologous region in one DNA strand with the trailing homologous region in another. As a result, one daughter cell contains two copies of the cassette. This process is repeated as long as recA is present. FIG. 1C depicts how a chromosome is evolved to high gene copy number by selection on antibiotics. As selection pressure increases, only cells with many CIChE duplications survive. recA, which is required for the chromosome evolution, is deleted after the cell has high gene copy number. Upon recA deletion, no selection pressure is required to maintain the recombinant alleles.

FIG. 2 presents a schematic and a graph showing that allele segregation indicates that random distribution, not mutation rates, result in rapid productivity loss in plasmids. FIG. 2A depicts an allele segregation mechanism: (i) a DNA mutation eliminates proper expression of the gene(s) of interest while not affecting the selectable marker; (ii) plasmids are copied before cell division. This may occur as ordered replication (as shown), where each plasmid is replicated once, or random replication, where replication initiation is random and a plasmid may be replicated more than once; (iii) during cell division, plasmids are randomly transmitted, leading to a possibility that both daughter cells receive one mutant plasmid or one daughter cell receives both mutant plasmids. Multiple copies of a mutant plasmid can accumulate quickly, leading to a higher growth rate compared to cells with only unmutated plasmids. In this figure, a cell with one active plasmid is indicated as having fast growth and low productivity; a cell with two active plasmids is indicated as having medium growth and medium productivity, and a cell with three active plasmids is indicated as having slow growth and high productivity. X indicates a mutated allele. FIG. 2B shows a comparison of ordered versus random inheritance of plasmids. A subpopulation balance model revealed that random inheritance (allele segregation), not mutation rates, caused rapid plasmid productivity loss. If inheritance was forced to be ordered, the genetic stability was increased tenfold for the same mutation rate (see Example 5 for details of model).

FIG. 3 presents graphs showing that in the absence of antibiotics, gene copy number in CIChE recA− cells is more stable than in plasmid-bearing or CIChE recA+ cells. FIG. 3A shows gene copy numbers before and after seven rounds of subculture without the antibiotic chloramphenicol (cat) for identical constructs of cat that were expressed as multiple copies on a pBR322-based plasmid (plasmid recA+), multiple tandem genes in a recA+ strain (CIChE recA+) or multiple tandem genes in a recA− cell (CIChE recA−). In FIG. 3B, all strains contain cat and the PHB operon. This substantially increases the metabolic burden for maintaining each copy of the PHB operon, resulting in a greater growth advantage for losing copies. FIG. 3C shows PHB accumulation for strains in FIG. 3B before and after seven rounds of subculturing. CIChE recA− constructs were stable for the length of the experiment in all cases. Gene copy numbers were measured by qPCR on purified DNA before and after the subculturing. DCW, dry cell weight. Error bars are s.e.m. n=4.

FIG. 4 presents graphs showing that gene copy number and yield in CIChE strains meet or exceed plasmid counterparts. FIG. 4A depicts gene copy number of a CIChE construct. Cells were evolved in the presence of chloramphenicol; recA was deleted; and gene copy number was measured for two colonies from the deletion plate by qPCR. Gene copy number in CIChE strains reached the copy number of a medium-high copy plasmid, and copy number could be controlled by the chloramphenicol concentration used in evolution. FIG. 4B shows PHB accumulation of CIChE constructs. Yield increased as more chloramphenicol was used in the chromosomal evolution until yield reached levels equivalent to those of an analogous plasmid-bearing strain. CIChE-PHB strains were from FIG. 4A and were grown without antibiotics. Plasmid-bearing strain used pTGD-PHB with a pBR origin and was grown with antibiotics. FIG. 4C shows that lycopene yields with CIChE constructs exceed equivalent plasmid systems. Lycopene yield increases with chloramphenicol concentration used in the chromosomal evolution. Plasmid-bearing strain used pAC-LYC with a p15A origin and was grown on antibiotics. DCW, dry cell weight. Error bars are s.e.m. n=4. Two colonies were analyzed from each chloramphenicol concentration.

FIG. 5 presents graphs showing that CIChE improves genetic stability. Strains bearing CIChE constructs, cultured without antibiotic selection, maintained PHB productivity for >35 generations longer than those bearing an equivalent plasmid, cultured with antibiotics. FIG. 5A shows that growth rate is substantially hindered by PHB production. After ˜35 generations, the plasmid system increased growth rate to the maximal growth rate, implying that the metabolic load of PHB production had been lost. CIChE strain does not change growth rate over the course of the experiment. Maximal growth rate is defined as strain without PHB production. FIG. 5B shows that CIChE PHB-specific productivity is higher, and lasts longer than that of plasmid system. Initial PHB productivity of the plasmid system was fourfold lower than CIChE. After 35 generations, plasmid productivity was completely lost. FIG. 5C shows the fraction of cells that made PHB. In plasmid-based PHB production, a nonproductive subpopulation overtook the culture with time. CIChE cells had a homogeneous population of cells making PHB. ‘Percentage cells producing PHB’ (y axis) is fraction of cells in population with PHB-associated fluorescence higher than fluorescence of a non-PHB-producing control (P<0.05). Generations based on the doubling time of the parent strain. Error bars are s.d. n=2.

FIG. 6 is a schematic depicting tandem gene duplication: construction, amplification, and stabilization. FIG. 6A shows a construct being delivered to the genome that contains an antibiotic marker and the gene(s) of interest flanked by homologous regions; FIG. 6B shows that recA mediates an uneven homologous crossover between regions flanking the antibiotic marker and gene of interest; FIG. 6C shows that this generates a strand with two copies of the cassette and another with a deletion; FIG. 6D shows that one daughter cell inherits the two repeats and the other daughter cell has lost the insert; FIG. 6E shows that antibiotics are used to provide a growth advantage for the cell with increased repeats. The process can then repeat itself to further amplify the number of duplications; FIG. 6F shows that finally, recA is deleted to prevent further change in copy number. [A] Homologous region (1 kb of chlB from Synechocystis PCC6803); [B] Antibiotic Resistance Gene (chloramphenicol acetyl transferase); [C] Gene of Interest (e.g., polyhydroxybutyrate operon or lycopene operon).

FIG. 7 is a schematic showing how tandem genes maintain alleles by avoiding ‘allele segregation.’ DNA replication errors that eliminate recombinant expression occur infrequently at ˜1 in 10⁹ bases copied [A]. Copies of a mutant allele accumulate quickly from plasmid expression due to ‘allele segregation.’ By this, after a mutated plasmid is copied, both copies of a mutant allele could be inherited by one daughter [B]. As the mutant allele copy number increases, so does growth rate, due to loss of recombinant expression. Because tandem genes inheritance is ordered by being on the genome, mutant alleles can not accumulate in one daughter cell [C], resulting in significantly extended allele expression. In this example 3 copies exist either as a plasmid or tandem genes, with 1, 2, or 3 mutant alleles present. Mutant fractions are calculated as the expected number of each mutant copy number based on binomial distributions and differences in growth rates. Generations based on doubling time of slowest strain.

FIG. 8 is a schematic depicting a map of plasmid pTGD (5628 bp). Features shown include a pBR322 origin of replication, a gene encoding β-lactamase (Amp^{R}) for ampicillin resistance, two identical chlB sequences flanking a gene encoding chloramphenicol acetyl transferase (Cm^{R}) for chloramphenicol resistance and a multiple cloning site. The chlB sequences are 1 kb non-coding regions from the middle of light-independent protochlorophyllide reductase subunit B of Synechocystis PCC6803. pTGD is used directly with the λInCh integration system for delivery to the genome.

FIG. 9 presents schematics of metabolic pathways. FIG. 9A depicts the PHB pathway from acetyl-CoA. FIG. 9B depicts a non-mevalonate pathway for lycopene.

FIG. 10 presents graphs depicting CIChE and plasmids in batch culture with and without antibiotics. Plasmid copy number decreases from stationary to growth phase. In a no glucose pre-innoculum, very high plasmid copy numbers were accumulated, but dropped upon culture in glucose. CIChE remained constant throughout. FIG. 10A presents a growth curve showing that CIChE constructs had a shorter lag phase than plasmid. Without wishing to be bound by any theory, this could be because initial PHB operon copy number is lower than plasmid. FIG. 10B shows that copy numbers drop precipitously for plasmids, while CIChE is constant throughout. Without antibiotics, plasmid copy number goes to zero, while copy numbers in plasmid drops to the level of CIChE. Increasing copy numbers of plasmids are not observed late in the culture because PHB operon has a large growth penalty for growth on glucose. Preinnoculum was grown in glucose-free LB. ˜30 copies appears to be the maximum amount cells can tolerate for growth on glucose. FIG. 10C shows that final PHB titers are similar between CIChE without antibiotics and plasmid with antibiotics. Plasmid without antibiotics produced almost no PHB. Strains, CIChE [K12 recA::kan selected on 1,360 μg/mL], Plasmid [XL-1 Blue recA− (pZE-tacpha)], were inoculated from a 5 mL LB+antibiotic saturated culture to A(600)=0.015. Cells were grown in 50 mL MR media at 37° C. and 225 rpm in duplicate. CIChE and Plasmid−antibiotic cultures had no chloramphenicol, while Plasmid+antibiotic had 34 μg/mL chloramphenicol. A(600), copy number, and PHB were determined as described. Two qPCR measurements were taken for each culture, total n=4. A(600) and PHB were n=2.

FIG. 11 is a graph depicting PHB production for TGD/plasmid strains. FIG. 11A shows % PHB (DCW), FIG. 11B shows PHB (mg/L), and FIG. 11C shows residual mass (mg/L). K12::PHBtac Cm20 ΔrecA produced high levels of PHB, equivalent to that of plasmids (both in this experiment and in the literature) in shake flasks. This strain also produced PHB much earlier in the growth process, and was able to produce this PHB without any antibiotics. The biomass grown was also highest with the TGD strain. XL1(pZE-tac-pha)+Cm produced PHB much later in the fermentation and was not able to achieve the concentrations of PHB that TGD did in the length of the experiment. XL1(pZE-tac-pha)−Cm produced only trace amounts of PHB, because the PHB operon was not maintained by antibiotics.

FIG. 12 is a graph depicting dilution rate vs. time for TGD chemostat.

FIG. 13 is a graph depicting total DCW (biomass+PHB) and optical density (OD) of TGD chemostat.

FIG. 14 is a graph depicting residual cell weight (biomass) and PHB concentration in bioreactor for TGD chemostat.

FIG. 15 is a graph depicting glucose and acetate concentrations in bioreactor for TGD chemostat.

### DETAILED DESCRIPTION OF THE INVENTION

Plasmids are an important tool for protein production and the expression of metabolic pathways in many organisms and are responsible for the manufacture of many specialty chemicals, biologics, small molecule drugs, and other materials. Plasmids are easy to transform into a cell and allow strong gene expression. While helpful, plasmids suffer from genetic instability that reduces the number of active recombinant alleles in a cell because of (1) segregational instability, plasmid-less cells resulting from unequal distribution of plasmids to daughter cells, and (2) structural instability, changes in the DNA sequence that cause incorrect expression of the desired protein. Because recombinant alleles typically reduce growth rate, both mechanisms give rise to subpopulations that grow faster and have fewer intact recombinant alleles than the parent cell, resulting in decreased productivity. Selectable markers, post-segregational killing (PSK) and partitioning elements help mitigate (1), and many cloning strains have implemented genotypes to reduce (2). Such methods have provided partial solutions by preventing plasmid-less population and slowing DNA mutations, but are suboptimal because these methods only imposes a minimum copy number, require expensive antibiotics, and can not prevent inevitable DNA replication errors. Genetic instabilities inherent in plasmid propagation have negatively impacted the development of continuous processes confining the majority of recombinant processes to batch operation.

Although methods have been devised to suppress (1) and (2), a third process, ‘allele segregation’ has not been described nor is prevented by existing methods, but will lead to a rapid loss in plasmid productivity. Plasmids typically containing a recombinant allele that expresses the gene-of-interest (called allele from here forward) and a selectable marker. Allele segregation occurs when a DNA mutation in the allele of a single plasmid in a multicopy plasmid system is quickly propagated to all copies in a cell, while the selectable marker remains intact. By this, unmutated plasmids are displaced, but mutated plasmids remain resistant to the selection pressure and confer a growth advantage by eliminating the allele expression. This results in a cell population that has lost the intended productivity, but is still correctly selected.

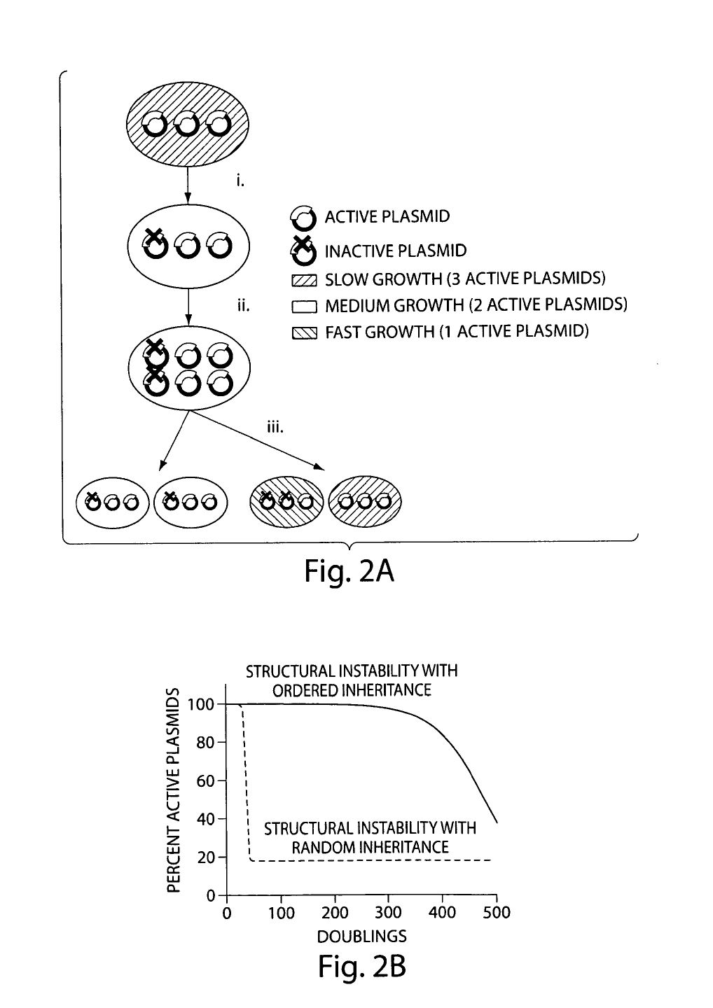

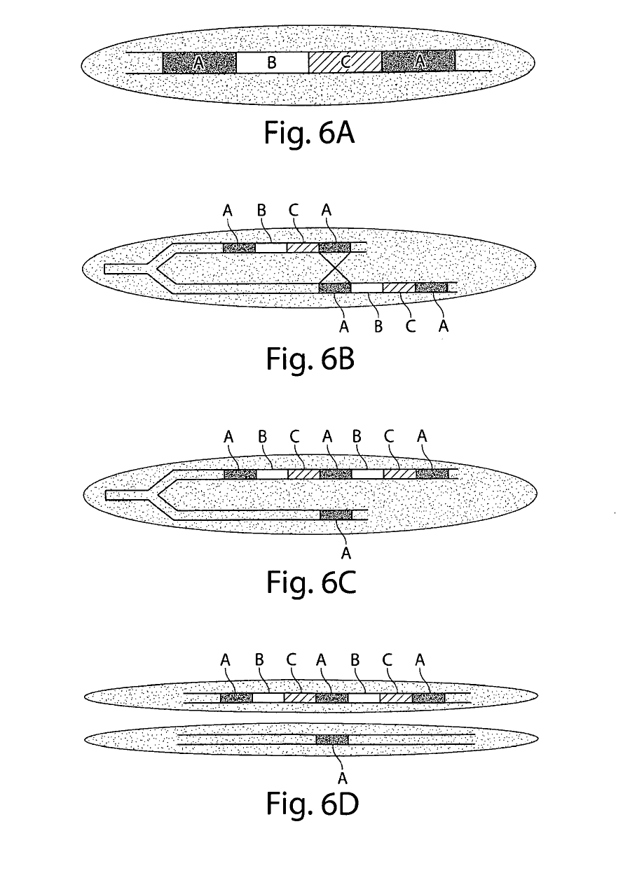

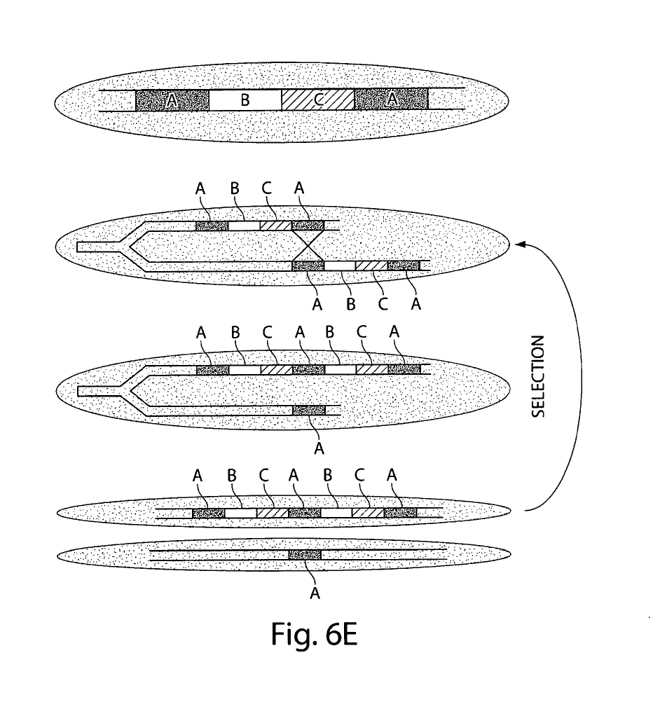

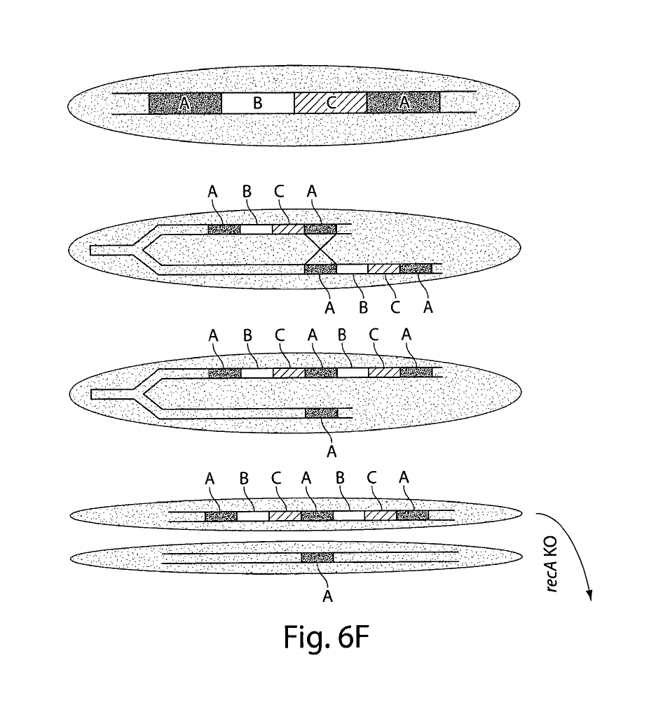

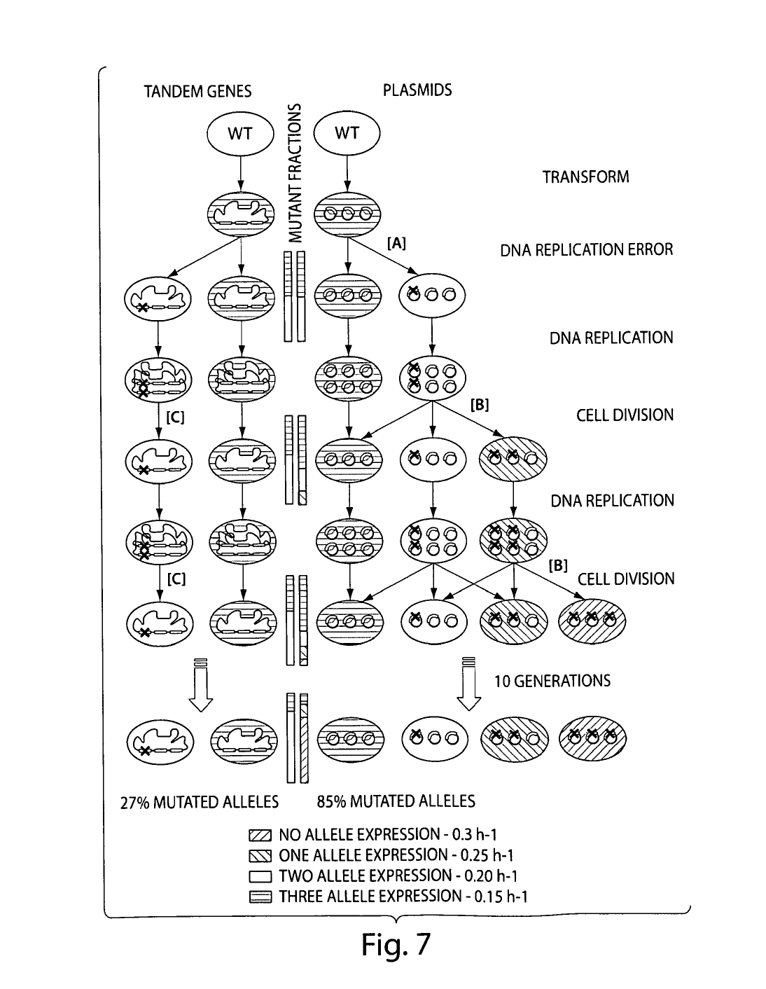

Allele segregation occurs due to the random distribution of plasmids from mother to daughter cells and the growth advantage conferred by loss of allele expression, as described. A mutation occurs in the allele of one copy of a plasmid among many non-mutated plasmids (FIG. 2[a]). The mutant plasmid will be copied (FIG. 2[b]), and upon cell division, one daughter cell can receive both mutant copies (FIG. 2[c]). If both mutant copies are received by one cell, it has effectively doubled its growth advantage. In subsequent generations, the cell with two mutant plasmids can give rise to three, four, etc., replacing unmutated plasmids while maintaining selectability (FIG. 6). par systems have some ability to partition to reduce the random distribution by partitioning copies to daughter cells, but has only shown partial success in plasmids with 1-5 copies/cell¹.

A simple model of plasmid propagation suggests that allele segregation decreases plasmid productivity longevity by 10 fold compared to DNA replication errors alone (FIG. 2[d]). For a typical medium copy plasmid system, a structural mutation will occur in 13 generations. If allele segregation can occur, this will result in a cell containing only mutant plasmids in 20 generations that will overtake the unmutated population in an additional 15 generations. In ˜50 generations then, allele segregation will lead to complete loss of plasmid productivity, all while maintaining the selectable marker. Without allele segregation, it would take ˜520 generations to lose plasmid productivity.

Tandem gene duplication (TGD) is a naturally occurring phenomenon that generates many head-to-tail repeats of a DNA sequence in the genome², giving rise to many copies of an allele that are not subject to allele segregation. TGD has been studied extensively in the context of evolutionary biology, but to date has not been shown to be genetically stable for recombinant expression^{3,4}. By avoiding allele segregation, engineered TGD can play a very important role in the introduction of new genes and pathways for long term cultivations, which is useful for example in impacting product formation in microorganisms and other application of metabolic engineering and synthetic biology.

Gene duplication events in TGD occur between two regions of high sequence homology surrounding a selectable marker (that will be used for the gene duplication, but not needed for fermentation) (see FIG. 6). The TGD mechanism causes gene duplication events to occur between two regions of high sequence homology driven and selected by a growth advantage conferred for each additional copy (FIG. 6(a)). An unequal crossover between the two homology regions on opposite strands (FIG. 6(b)) occurs after the replication fork. This crossover event is recA dependent in prokaryotes and yields two strands of DNA, one containing two copies of the region between the homologous sites and another with that region deleted (FIG. 6(c)). Each of these strands is passed down to one daughter cell (FIG. 6(d)). A growth advantage when grown on antibiotics is conferred by multiple copies of the selectable marker, causing the cell containing the tandem genes to overtake the population and be available for further rounds of duplication (FIG. 6(e)). This process can continue, and has been observed to make up to 200 copies of the inter-homologous region⁵. If conditions change such that a growth advantage is given for fewer copies, the copy number can be reduced by the same process. To prevent this, recA is deleted (FIG. 6(f)), thereby preventing the crossover step and fixing the copy number.

The engineering strategy reflecting one embodiment was to construct a DNA cassette containing gene(s) of interest and chloramphenicol acetyl transferase (cat), flanked on both sides by identical, non-coding 1 kb regions of foreign DNA that has low homology to any other region of the E. coli genome. The large identical regions served as homology regions for the crossover event, and increased tolerance to chloramphenicol was used to provide a growth advantage for cells containing duplicated genes. The construct was delivered to a wild type E. coli genome and subcultured in increasing concentrations of chloramphenicol. Once the cells have developed a resistance to the desired concentration of chloramphenicol and therefore have reached a desirable number of duplications, recA was deleted to prevent any further increase or decrease in copy number. At this point, the strain expressed the gene(s) according to the gene dosage and did not require the chloramphenicol to maintain the copy number. In this work we describe the methods to achieve the engineering strategy described above and present experimental evidence for the improved genetic stability that is achieved by tandem gene duplication and its application to metabolic engineering of the PHB pathway. The engineered tandem gene duplication approach developed herein is called “chemically inducible chromosomal evolution” (CIChE). The terms TGD and CIChE are used interchangeably herein to describe plasmid-free methods for engineering cells with high copy gene expression.

Tandem genes are an outstanding method for high copy expression in many respects. Most importantly, TGD avoids allele segregation, thereby significantly delaying the overtake by mutant alleles. This is achieved by linking all of the copies on a single strand of DNA in the genome. By linking them in the genome, each daughter cell is guaranteed to receive only one copy of each recombinant allele. Therefore, the mechanism for losing recombinant expression is by DNA mutations, which are much slower than allele segregation. Tandem gene-bearing cells can be propagated without any external antibiotic, and do not rely on auxotrophic markers or par elements. A key feature of this method is the use of recA as an ‘on/off’ switch to control the change in copy number of the tandem genes³. Without the deletion of recA, the utility of the TGD constructs would be reduced as copy number would decrease over time.

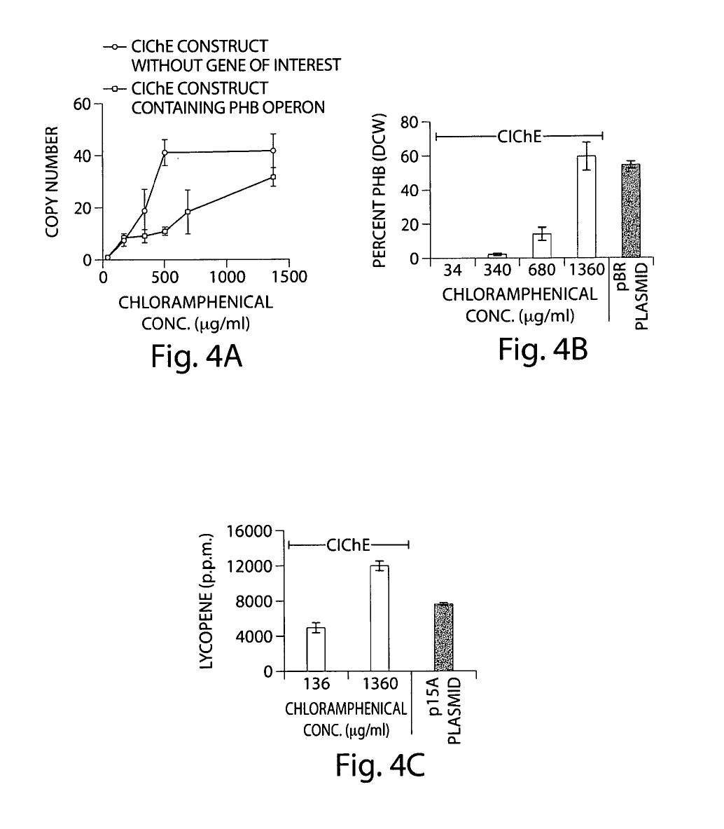

TGD is useful, for example, for synthetic biology and metabolic engineering. The copy number of the tandem genes is constant in a population, due to it being in the genome. This is compared to plasmids whose copy numbers are broadly distributed in a cell culture⁸. Additionally, plasmids with relaxed origins of replication can vary greatly in copy number between growth and stationary phase⁹. In synthetic control circuits, a transient variation in copy number could be problematic, leading to unintended responses and artifacts in the genetic circuit. Furthermore, the copy number in TGD can be tuned over a continuous range by the strength of antibiotic used in the gene amplification step as shown in FIG. 4. This is equivalent to tuning promoter strength and allows for a range of expression for a given gene by controlling the copy number.

Tandem gene constructs match the high gene dose of multicopy plasmids. Unlike other genomic integration approaches that are tedious to extend beyond one copy, the copy numbers of TGD can be varied over much of the same range as plasmids. This is shown both in the measurement of copy number and in the productivity of PHB and lycopene, verifying that high expression is possible. In one example, we were able to achieve PHB accumulation up to 70% PHB (DCW), on par with values measured using plasmids¹⁰. This is actually surprising, given that Lee et. al, were only able to get 22% PHB (DCW) with K12 E. coli, while XL-1 Blue produced 80% PHB (DCW)¹⁰. We hypothesize that plasmid expression problems limited PHB production in K12 that were overcome using TGD, boosting PHB three fold. In a second example, the yield of the nutraceutical lycopene was increased by 60% using methods described herein.

To date, allele segregation has been overlooked as a limiting phenomenon in the length of process productivity, but must be addressed to achieve long term expression. While minimal genome approaches promise increased genetic stability by removal of transposons, prophage, and other genetic loci that would otherwise increase mutation frequencies¹¹, these approaches will fail if plasmids are used to express key pathways. DNA mutations are inevitable with any DNA polymerase, even in minimal genomes, and allele segregation will occur by the same mechanisms for plasmids in synthetic cells as for plasmids in E. coli. If steps are not taken to avoid allele segregation, the advantages of minimal genome cells will have little effect on the longevity of plasmid expression in synthetic strains. However, multiple copies of the desired pathway in the genome, by TGD or other synthetic means, will be much more stable.

TGD has far reaching potential in many different biotech applications. TGD constructs could be particularly useful in the production of low-value added commodity chemicals for two reasons (1) high level, long term expression, and (2) no antibiotics. Continuous processes have inherent economic advantages over batch processes, but have not traditionally been used in the biotech industry because of issues with sterility and genetic mutation. TGD significantly extends the time that recombinant genes can be expressed in continuous systems and may prove useful in low value products such as biofuels. The absence of antibiotics helps economics both by avoiding purchase as well as unnecessary waste treatment to prevent the low level emission of antibiotics to the environment. TGD will also be helpful in many high value products, where toxic compounds are generated while making the product, leading to very slow growing strains. When growth rate is extremely hindered by a recombinant pathway, selection for allele segregation is very large, leading to rapid propagation of mutant alleles. TGD constructs, however, are not subject to allele segregation, and are more tolerant to reduce growth rates. Engineered TGD should be applicable in most host organisms, because natural TGD events are ubiquitous in nature. By delivering analogous DNA constructs and inactivating recA homologues (e.g., rad51 or Dmc1), tandem genes can be created and stabilized. We expect this method to have wide industrial application for recombinant expression because of the stability, high expression, and lack of need for plasmid maintenance.

The invention provides methods for producing a genetically stable tandem gene duplication. The methods include integrating into a chromosome of a host cell a nucleic acid construct comprising a nucleic acid sequence that encodes one or more proteins operably linked to one or more promoter sequences, a nucleic acid sequence encoding a selectable marker, and homologous nucleic acid segments flanking the nucleic acid sequences that encode the one or more proteins and the selectable marker, wherein the host cell comprises a functional recombinase. Second, the methods include selecting for tandem gene duplication (TGD) of the nucleic acid sequences that encode the one or more proteins and the selectable marker by culturing the host cell under selective conditions in which the selectable marker confers a growth advantage to the host cell, wherein the TGD is mediated by the functional recombinase. The number of copies of the nucleic acid sequences between the flanking homologous nucleic acid segments can be increased to at least 10, 15, 20, 25, 30, 40, 50, 60, 70, 80, 90, 100, 110, 120, 130, 140, 150, 160, 170, 180, 190, 200 copies or more. Third, the methods include stabilizing the TGD by deleting the recombinase or disabling the recombinase.

An important feature of the invention is the inactivation of recombinase once a desired amplification of the gene of interest is achieved to stabilize the tandem gene duplication. Typically, the recombinase is encoded by the host cell. As shown herein, recA of E. coli host cells was deleted to inactivate its activity and stabilize the TGD. Synonyms for recA include EG10823, lexA, lexB, recH, rnmB, srf, tif, umuB, and zab; see Hoffmann, R., Valencia, A. A Gene Network for Navigating the Literature. Nature Genetics 36, 664 (2004) and the iHOP website at ihop-net.org. It also is possible to inactivate recA function by other methods known in the art, such as single or multiple mutations, partial deletion, etc. In other host cells, the active recombinase(s) can be inactivated by deletion or mutation in a similar fashion in order to inactivate the recombinase(s) and thereby stabilize the TGD. Stabilizing the TGD provides for long term stable expression of the gene of interest in the TGD.

Certain constructs may include a recombinase system, and care must be taken to remove such sequences to preserve stability of tandem gene duplications produced using such constructs. For example, it is possible to use Lambda phage to deliver the gene of interest to the genome in the manner described herein. However, unless the phage is subsequently removed, the tandem gene duplications will not be stable because the phage has its own recombinase system.

Integration of the TGD construct into a host cell can be carried out using any methods known in the art. Such methods include the Lambda phage-based λInCh protocol described by Boyd et al. (J. Bacteria 182, 842-847 (2000)). This protocol delivers the plasmid construct to the genome by site specific recombination of the phage into the 1 att site followed by a recombination step that removes the lambda phage DNA from the genome. Other methods known in the art also can be used, including integrating a PCR product (such as in Datsenko K. A. & Wanner B. L. One-step inactivation of chromosomal genes in Escherichia coli K-12 using PCR products. Proc Natl Acad Sci USA. 97, 6640-5 (2000)). Such methods can also facilitate delivery to the genome of the promoter and/or coding sequence of the TGD construct in a second step.

The methods and constructs described herein are in some embodiments used with prokaryotic host cells. Among prokaryotic hosts, gram negative bacteria are preferred, especially Escherichia coli. Other prokaryotes, such as Bacillus subtilis or Pseudomonas may also be used.

Naturally occurring tandem genes have been observed in eukaryotic cells, and recA homologues exist in eukaryotic cells. Thus, while the embodiments described herein include prokaryotic cells as host cells, the constructs and methods described herein can also be used in eukaryotic systems, particularly single celled systems. The particular flanking sequences, selectable markers, and promoters can be varied according to the host cell used. The recombinase system used by the host cells (which may include more than one type of recombinase) can be inactivated by deletion or mutation using standard methodologies.

Mammalian or other eukaryotic host cells, such as yeast, filamentous fungi, plant, insect, amphibian or avian species may also be used. Mammalian host cell lines include, but are not limited to, VERO and HeLa cells, Chinese hamster ovary (CHO) cells, and W138, BHK, and COS cell lines.

A variety of different proteins of interest can be encoded in the constructs described herein and produced by the methods described here. The type of protein is essentially not limited. In some embodiments, the protein is a protein that is non-native to the host cell, but native proteins of the host cell also can be produced, such as for optimizing expression of a protein or proteins native to the host cell. Likewise, the number of proteins is not particularly limited by the constructs and methods of the invention other than by any inefficiencies in tandem gene duplication with long nucleic acid sequences that encode a large number of proteins. Two examples of constructs encoding multiple proteins are provided herein. The phaECAB operon was introduced into a TGD construct and this construct was used to produce cells with tandem gene duplications and high-level, stable expression of the proteins encoded by the operon, as evidenced by high-level, stable production of PHB. Additionally, the CrtEBI and dxs-idi-ispDF operons were used to produce cells with tandem gene duplications and high-level, stable expression of the proteins encoded by the operon, as evidenced by high-level, stable production of lycopene.

Conveniently, the nucleic acid sequence that encodes the one or more proteins is inserted in the TGD construct in a multiple cloning site that is located between the flanking homologous nucleic acid segments. Production of such multiple cloning sites is well known in the art.

Typically, increasing the number of copies of the nucleic acid sequence encoding the selectable marker confers increasing growth advantage to the host cell. The selectable marker can be any that gives the cell a growth advantage by having multiple copies. Thus in some embodiments, the step of selecting for TGD includes successive rounds of culture of the cell under culture conditions that successively require an increase in the number of copies of the nucleic acid sequence encoding the selectable marker. This is demonstrated in the Examples by culturing in successively increasing amounts of chloramphenicol, with progressive increases in the number of copies of the chloramphenicol resistance gene and the gene of interest in between the flanking homologous nucleic acid sequences. In some embodiments, the nucleic acid sequence encoding the selectable marker is an antibiotic resistance gene, such as a chloramphenicol resistance gene or a tetracycline resistance gene. In such embodiments, the selective conditions include culturing the cells in medium that contains the corresponding antibiotic, such as chloramphenicol or tetracycline. In other embodiments, the nucleic acid sequence encoding the selectable marker is a gene encoding a protein that is required for producing a nutrient required by the host cell to complement auxotrophic deficiencies, e.g., an auxotrophic marker gene. In such embodiments, the selective conditions include culturing the cells in a medium that does not supply the metabolite produced by the auxotrophic marker gene.

The flanking homologous nucleic acid segments of the TGD constructs are sufficiently non-identical with the genome of the host cell that they cannot efficiently recombine with the genome of the host cell in the presence of the functional recombinase, and are sufficiently homologous with each other and of sufficient length that they can recombine efficiently with each other in the presence of the functional recombinase. The length and amount of homology of the homologous nucleic acid segments will influence the efficiency of duplication of the nucleic acid sequence that they flank. In some embodiments, the flanking homologous nucleic acid segments are at least 25 nucleotides in length. More preferably, the flanking homologous nucleic acid segments are at least 50, 100, 200, 300, 400, 500, 600, 700, 800 or 900 nucleotides in length. More preferably still, the flanking homologous nucleic acid segments are at least 1000 nucleotides in length. In other embodiments, the flanking homologous nucleic acid segments are less than: 50%, 40%, 30%, 20%, 10%, 5%, 4%, 3%, 2%, or 1% identical with the genome of the host cell. In other embodiments, the homologous nucleic acid segments are at least 50% identical, more preferably at least 60%, 70%, 80%, 90% or 95% identical (to each other). More preferably still, the homologous nucleic acid segments are identical. In certain embodiments, there are two homologous nucleic acid segments flanking the nucleic acid sequences that encode the protein and the selectable marker.

In some embodiments, the flanking homologous nucleic acid segments are derived from a genome other than that of the host cell. In certain embodiments, the host cell is a bacterial cell and the flanking homologous nucleic acid segments are derived from a genome of a different species of bacteria or a eukaryotic cell genome. In one embodiment, the host cell is an E. coli cell and the flanking homologous nucleic acid segments are derived from a Synechocystis genome. Additional non-host cell nucleic acids will be known to the person of skill in the art upon selection of the host cell to be used. In some embodiments, the flanking homologous nucleic acid segments are non-coding.

One of the advantageous features of the invention is the stability of the tandem gene duplication over many generations without selection for its maintenance in the genome of the host cell. In some embodiments, the TGD is stable over more than 50, more than 60, more than 70, more than 80, more than 90, or more than 100 generations. Thus, the host cell need not be cultured under selective conditions after the tandem gene duplication is stabilized by deleting the recombinase or disabling the recombinase.

Promoter sequences of any kind known in the art can be used in the TGD constructs and methods. However, in some embodiments, the one or more promoters used is/are dependent on the native RNA polymerase of the host cell.

According to another aspect of the invention, cells produced by the method of any of the foregoing methods are provided. Also provided are containers containing a plurality of such cells, and cell cultures of such cells. Such cells can be stored and used in the manner that cells ordinarily are.

In particular, the cells can be used to start production cultures for producing proteins or metabolites. Thus the invention also provides methods for producing a protein or metabolite. The methods include culturing a cell produced by any of the foregoing methods in culture medium. In some embodiments, the methods further include isolating and/or purifying the protein or metabolite from the cell or culture medium using any of the methods well known to the person of skill in the art. In some embodiments, the protein or metabolite is a nutraceutical such as lycopene. As used herein, a nutraceutical refers to a substance that may be considered as a food or dietary supplement to improve the diet or provide therapeutic benefit.

Nucleic acid constructs also are provided that are useful, inter alia, in the methods described herein. The nucleic acid constructs include a nucleic acid sequence that encodes one or more proteins operably linked to one or more promoter sequences functional in a host cell, a nucleic acid sequence that encodes a selectable marker, and homologous nucleic acid segments flanking the nucleic acid sequence that encodes the one or more proteins and the nucleic acid sequence that encodes the selectable marker. The nucleic acid sequence that encodes the one or more proteins is operably linked to the one or more promoter sequences on a multicopy number plasmid vector having an origin of replication during construction of the construct. In some embodiments, the origin of replication is a bacterial origin of replication.

The invention also provides cells that include the foregoing nucleic acid construct. Also provided are containers containing a plurality of such cells and cell cultures of such cells.

General techniques for nucleic acid manipulation useful for the practice of the claimed invention are described generally, for example, in Sambrook et al., MOLECULAR CLONING: A LABORATORY MANUAL, Vols. 1-3 (Cold Spring Harbor Laboratory Press, 2 ed., (1989); or F. Ausubel et al., CURRENT PROTOCOLS IN MOLECULAR BIOLOGY (Green Publishing and Wiley-Interscience: New York, 1987) and periodic updates. Reagents useful in nucleic acid manipulation, such as restriction enzymes, T7 RNA polymerase, DNA ligases and so on are commercially available.

A “foreign”, “non-native” or “heterologous” polypeptide is a polypeptide which is not normally found in a host cell of a particular species. The nucleic acid encoding such a polypeptide is also referred to as “foreign” or “heterologous.” The heterologous proteins may be of mammalian, other eukaryotic, viral, bacterial, cyanobacterial, archaebacterial, or synthetic origin. A “non-bacterial protein” is a foreign or heterologous protein or polypeptide which is not naturally found in a bacterial cell. Non-bacterial proteins include viral and eukaryotic proteins. Non-bacterial, foreign, or heterologous proteins may also be fusions between non-bacterial, foreign, or heterologous proteins and other proteins or polypeptides. A “native” polypeptide or DNA sequence, by contrast, is naturally found in the host cell. For the embodiments encompassed by this invention, any of the foregoing types of polypeptides may be expressed.

“Nucleic acid sequences encoding heterologous, foreign or non-bacterial proteins” contain all of the genetic elements necessary for the expression of the heterologous, foreign or non-bacterial proteins, optionally with the exception of a promoter functional in the host cell. These sequences encompass recombinant genes which may include genetic elements native to the host cell. Further, the coding regions of these genes may optionally be optimized for the codon usage of the host cell.

A nucleic acid sequence “encodes” a polypeptide if, in its native state or when manipulated by recombinant DNA methods, it can be transcribed and/or translated to produce the polypeptide.

A nucleic acid sequence is “operably linked” when it is placed into a functional relationship with another nucleic acid sequence. For example, a promoter is operably linked to a coding sequence if the promoter affects its transcription or expression. Generally, DNA sequences which are operably linked are contiguous and, where necessary, in reading frame.

An appropriate promoter and other sequences necessary for efficient transcription and/or translation are selected so as to be functional in the host cell. Examples of workable combinations of cell lines and expression vectors are described in Sambrook et al., supra or Ausubel et al., supra. Promoters such as the trp, lac and phage promoters (e.g., T7, T3, SP6), tRNA promoters and glycolytic enzyme promoters are useful in prokaryotic hosts.

In some embodiments, a promoter that is dependent on the host cell native RNA polymerase is used. Because the native RNA polymerase is required for cell function, the promoter can not easily be mutated, leading to silencing of the promoters used in the methods and products described herein, without broader deleterious effects on the cell. Certain promoters, such as the T7 promoter, require a special RNA polymerase that is not native to the host cell. Such promoters are not used in some embodiments because the promoter function will not be stable because the T7 RNA polymerase can be easily inactivated, which would silence all the T7 promoters in the tandem genes.

Useful yeast promoters include the promoter regions for metallothionein, 3-phosphoglycerate kinase or other glycolytic enzymes such as enolase or glyceraldehyde-3-phosphate dehydrogenase, enzymes responsible for maltose and galactose utilization, and others. Appropriate mammalian promoters include the early and late promoters from SV40 or promoters derived from murine Moloney leukemia virus, mouse mammary tumor virus, avian sarcoma viruses, adenovirus II, bovine papilloma virus or polyoma virus.

A “recombinant” nucleic acid is one that is made by the joining of two otherwise separated segments of nucleic acid sequence in vitro or by chemical synthesis.

“Chromosomal amplification” refers to the increase in copy number of a DNA sequence on the host cell chromosome.

Nucleic acid probes and primers are isolated nucleic acids, optionally single stranded, and, especially in the case of probes, are typically attached to a label or reporter molecule. Probes are used, for example, to identify the presence of a hybridizing nucleic acid sequence in a tissue or other sample or a cDNA or genomic clone in a library. Primers are used, for example, for amplification of nucleic acid sequences, e.g., by the polymerase chain reaction (PCR). The preparation and use of probes and primers is described, e.g., in Sambrook et al., supra or Ausubel et al. supra, or may be chemically synthesized using methods well known in the art.

The constructs used in the methods described herein include a selectable marker, which is a gene encoding a protein necessary for the survival or growth of a host cell transformed with the construct. Typical selectable markers (a) confer resistance to antibiotics or other toxic substances, e.g. chloramphenicol, ampicillin, neomycin, methotrexate, etc.; or (b) complement auxotrophic deficiencies. The choice of the proper selectable marker will depend on the host cell. The presence of the selectable marker in the construct permits selective pressure to be exerted on the host cell, which causes tandem gene duplication of the construct as described herein.

A variety of methods for introducing nucleic acids into host cells are known in the art, including, but not limited to, electroporation; transfection employing calcium chloride, rubidium chloride calcium phosphate; DEAE-dextran, or other substances; microprojectile bombardment; lipofection; and infection (where the vector is an infectious agent, such as a retroviral genome). See generally, Sambrook et al., supra and Ausubel et al., supra.

The constructs and methods described herein can be used to increase expression of single or multiple proteins, for production of the single or multiple proteins, or to increase production of metabolites that are produced by the single or multiple proteins. This may take the form of tandem duplication of a whole metabolic pathway, or a part of such a pathway, as exemplified for the PHB pathway herein. Following expression of the single or multiple proteins, the protein product(s) can be isolated and/or purified. As will be apparent to one skilled in the art, the isolation and/or purification method(s) used will depend on the identity of the protein(s).

Advantageously, no antibiotics are required in culturing the host cells, because the TGD is stable and does not require continual selective pressure of antibiotics to maintain the presence of the construct.

Advantageously, host cells modified to contain TGDs as described herein can be used in continuous reactors (e.g., chemostats) as demonstrated herein.

Promoter engineering, which may be considered the ability to tune promoter strength, is a useful tool. Promoter engineering can be accomplished by varying copy number of the tandem genes in accordance with the methods described herein, as is exemplified below.

The methods and constructs of the invention also can be used to have “ordered inheritance” of gene copies. This arises from the stability of the TGD in the genome, in contrast to allele segregation wherein multicopy plasmids are inherited randomly.

The methods and constructs of the invention also can be used in synthetic biology where copy number is advantageously kept constant. Plasmid copy number can vary in a population and in time (growth/stationary phase), which can cause problems for control circuits in the cell. In contrast, the present methods and constructs facilitate stable, tunable expression of sequences.

The invention will be better understood by reference to the following examples, which are intended to merely illustrate the invention. The scope of the invention is not to be considered limited thereto.

### EXAMPLES

### Materials and Methods

### Strains and Media

E. coli K12 was used for the host strain of all tandem gene duplications and plasmid comparison experiments. XL-1 Blue (Stratagene) was used for long term plasmid comparison experiment. DH5α (Invitrogen) was used for cloning steps. Lysogens and transfer strains used in the λInCh protocol were kindly provided by Dr. Dana Boyd and Dr. Jon Beckwith⁶. BW 26,547 recA::kan Lambda recA+, generated by Barry Wanner, used for P1 phage transduction of the recA deletion allele to the TGD strains, was received from Bob Sauer. Genomic DNA was isolated from Synechocystis PCC6803 using the Wizard Genomic Purification kit (Promega). pAGL20, a modification of pJOE¹² is a gift from Anthony Sinskey, containing the contains the genes phaAB from Ralstonia eutropha, encoding the β-ketothiolase and the acetoacetyl coenzyme A reductase, and phaEC from Allochromatium vinosum, encoding the two-subunit PHB polymerase. pZE-tac-pha is subcloned from pZE21¹³, with the PHB operon from pAGL20 and a P_{tac} promoter (described further in Example 2). Luria-Bertani broth was used for routine culturing and for tandem gene amplification. PHB production was measured when grown in minimal MR medium¹⁴ with 20 g/L glucose.

### Construction of TGD Strain

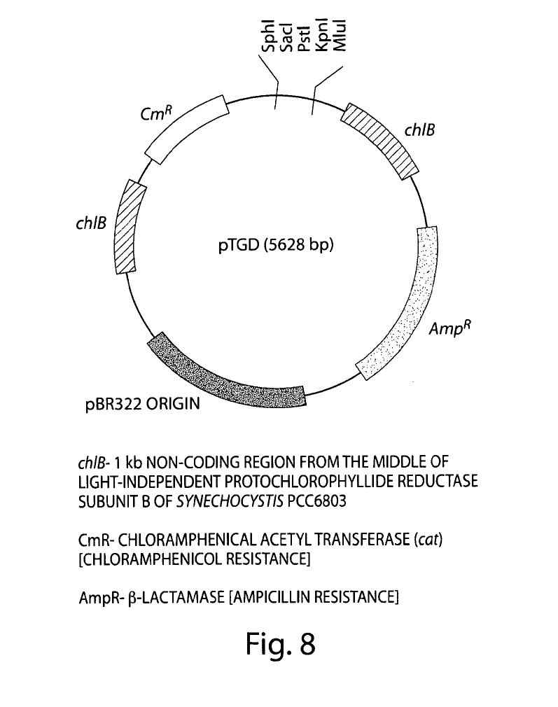

All oligonucleotides used in cloning are shown in Table 1. pTrcHis2B (Invitrogen) was digested by SphI & ClaI, blunted by Mung Bean Nuclease and ligated to yield only the pBR322 origin and β-lactamase. Overlap PCR¹⁵ was used to connect a I kb region of chlB from Synechocystis PCC6803 to the chloramphenicol acetyl transferase (cat) from pAC-LYC. The resulting DNA, followed by another copy of the chlB were sequentially cloned into the multicloning site of the pTrcHis2B-derived plasmid. This plasmid, pTGD, was used as a general integration platform (Plasmid map in FIG. 8). pTGD-PHB was created from the phaECAB operon with tac promoter and optimal RBS from pZE-tac-pha cloned into the SphI/MluI sites of pTGD. The resulting DNA constructs were transferred from the plasmids to the genome of K12 E. coli using the λInCh protocol⁶. This protocol delivers the plasmid construct to the genome, followed by a recombination step that removes the lambda phage DNA from the genome.

Amplification of the construct was accomplished by subculturing the resulting strains in LB (100× dilution), doubling the chloramphenicol concentration each time from 13.6 μg/mL to the desired concentration (as high as 1,360 μg/mL). recA deletion was accomplished by P1 phage transduction of the recA::kan allele to the TGD strain. recA− was routinely validated by UV sensitivity to 3,000 μJ energy using a Stratalinker UV crosslinker (Stratagene, La Jolla, Calif.).

### Subculturing Genetic Stability Assay

Strains were grown in 5 mL LB in 14 mL culture tubes at 37° C. without antibiotics. After cultures reached stationary phase (typically ˜12 hrs), 50 μL were diluted into a fresh 5 mL of LB (100× dilution). This was repeated a total of seven times. At this point, genomic DNA was purified and copy numbers measured by qPCR. A sample of the cells was grown in MR medium for 3 days and PHB analyzed.

### Exponential Phase Genetic Stability Assay

Cells were cultured by continuous subculture, as in the cells were not allowed to enter stationary phase. Cells were grown in 250 mL Erlenmeyer shake flasks with 50 mL MR media at 37° C. and 225 rpm. Cells were inoculated at A₆₀₀=0.015 and were allowed to grow to late exponential phase (typically A₆₀₀=2.0). Cells were subcultured by inoculating the culture to A₆₀₀=0.015 in a prewarmed shake flask as above. By this, cells were continuously growing at maximal growth rate and did not enter stationary phase during the course of the experiment. Specific growth rate was estimated in each subculture based on the A₆₀₀ data points taken. PHB accumulation was measured as below and used to calculate PHB productivity as the product of specific growth rate and PHB accumulation, an approximation that holds at steady state.

qPCR Measurement of Copy Number

Copy numbers were detected by qPCR on genomic DNA isolated from the appropriate strains using the Wizard Genomic Purification kit (Promega). qPCR was carried out on a Biorad iCycler using the iQ SYBR Green Supermix (Biorad). cat copy numbers was detected and compared to the copy number of bioA, a nearby native gene in the genome. Table 1 has primers used for qPCR. Genomic DNA containing only one copy of the construct was diluted and used as a standard curve.

Poly-3-hydroxybutyrate Assay

End point PHB accumulation of the strains was determined by inoculating an overnight culture of LB (1% v/v) into a 250 mL shake flask with 50 mL of MR media. Cells were cultured at 37° C. for 72 hrs at 225 rpm. Cells were harvested by centrifugation, and PHB content was analyzed by hydrolysis to crotonic acid followed by HPLC analysis as described elsewhere¹⁶. An authentic PHB standard was purchased from Sigma-Aldrich.

### FACS Assay

Percentage of cells producing PHB was determined by fluorescence activated cell sorting (FACS) using nile red to stain the PHB granules¹⁶. Samples were collected from the exponential phase subculturing experiment, and allowed to grow to late stationary phase (to maximize the PHB in a cell). 200 μL of cell culture was mixed with 800 μL isopropanol to fix the cells. This was incubated for 10 min and then resuspended in 10 mg/mL MgCl with 3 μg/mL nile red. Cells were stained with nile red for 30 min in the dark and analyzed on a Becton Dickinson FACScan. Cells were scored as containing PHB if the fluorescence was above the 95^{th} percentile of a distribution of cells containing no PHB.

### Example 1

### Introduction

TGD was implemented by constructing a DNA cassette containing gene(s) of interest and chloramphenicol acetyl transferase (cat), flanked on both sides by identical, non-coding 1 kb regions of foreign DNA that has low homology to any other region of the E. coli genome. The large identical regions served as homologous substrates for the crossover event, and increasing chloramphenicol concentration was used to select for cells containing duplicated genes. The construct was delivered to a wild type E. coli genome and subcultured in increasing concentrations of chloramphenicol. Once the cells had developed resistance to a particular concentration of chloramphenicol and therefore had reached a desirable number of duplications, recA can be deleted to prevent any further increase or decrease in copy number. After this, the strain expressed the gene(s) according to the gene dosage and did not require chloramphenicol to maintain the copy number. In this work we describe the methods to implement TGD and present experimental evidence for the improved genetic stability that is achieved by TGD and its application to metabolic engineering of the PHB pathway. We show this concept was successful in allowing cells with heavy metabolic burdens to persists for significantly longer than analogous plasmids, thus opening up the possibility for the broad use of TGD-engineered microbes in large scale industrial production.

### Results

A shuttle plasmid, pTGD, was constructed to deliver the required DNA elements to the genome using the λInCh genomic integration system⁶ (details in Materials and Methods). pTGD contains two identical, non-coding 1 kb regions of Synechocystis DNA flanking cat and a multicloning site. Following delivery to the genome of E. coli, the copy number was amplified by serial subculturing in increasing concentrations of chloramphenicol, selecting for cells with more tandem copies (called TGD(cat)). In equivalent recA− strains, increased resistance to chloramphenicol could not be induced by serial subculture, implying that tandem repeats could not be generated without recA (data not shown). A second TGD strain was created with the PHB operon cloned into the multicloning site of pTGD using the methods above (called TGD(cat+PHB)). While λInCh was chosen to integrate the TGD cassette in this study, a variety of other methods well known to the person of ordinary skill in the art could be used to introduce the unamplified construct to the cell.

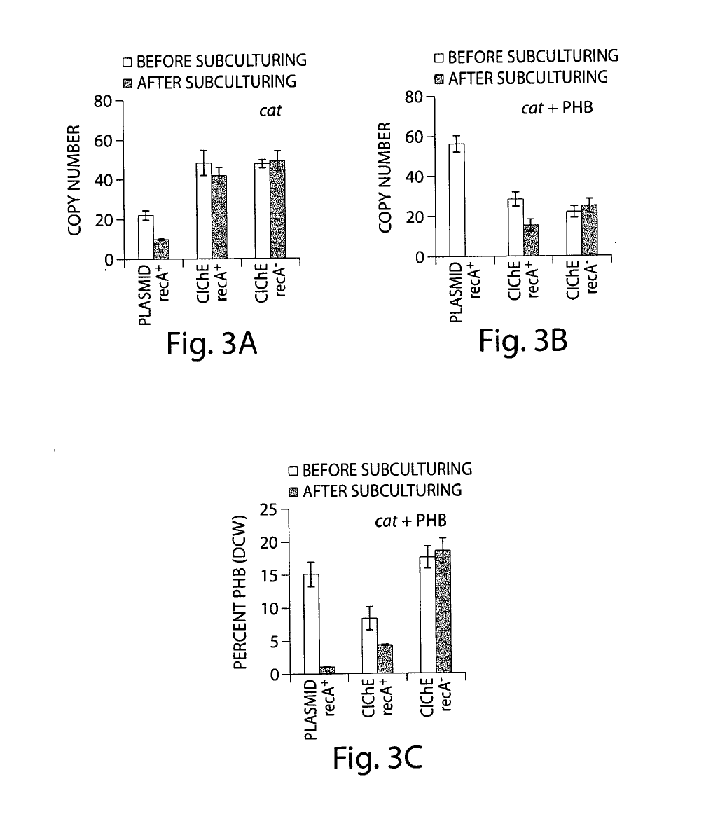

TGD constructs were first compared to the copy number stability of plasmid segregation without antibiotics. TGD (cat) recA± and K12 (pTGD), a plasmid version of the TGD cassette, were subcultured seven times (˜45 generations) (FIG. 3(a)). As expected, the plasmid copy number was significantly reduced. Tandem gene constructs, both recA+ and recA−, faired much better and did not have reduced copy number. Subsequently we examined genotypes that would place a large metabolic burden on the cell by expressing the resource-requiring recombinant PHB pathway. These conditions result in a growth disadvantage of the PHB-containing strain that would normally lead to a drastic plasmid copy number reduction when subject to the same subculturing as above. This is what is observed as the plasmid copy number had been reduced by 99.9% (FIG. 3(b)). The TGD (cat+PHB) recA+ strain copy number was also reduced, due to possible DNA crossover that would reduce copy number. However, the reduction was not as dramatic as with the plasmid because DNA crossover, which occurs in ˜7 generations for high homology regions of TGD⁷, is not expected to be as frequent as segregation instability. The TGD (cat+PHB) recA− strain maintained its copy number, even in the presence of selection pressure imposed by the PHB operon. The ability of recA to act as an ‘on/off’ switch for changing copy number represents an important control of gene copy number. PHB accumulation in late stationary phase tracked well with the copy number (FIG. 3(c)) verifying that active enzymes were being expressed to accumulate PHB. The plasmid bearing strain produced almost no PHB after subculture, while the TGD(cat+PHB) recA− construct produced the same amount of PHB as before subculture. This confirms that the gene copies are both present and active after many rounds of subculture in TGD recA− strains.

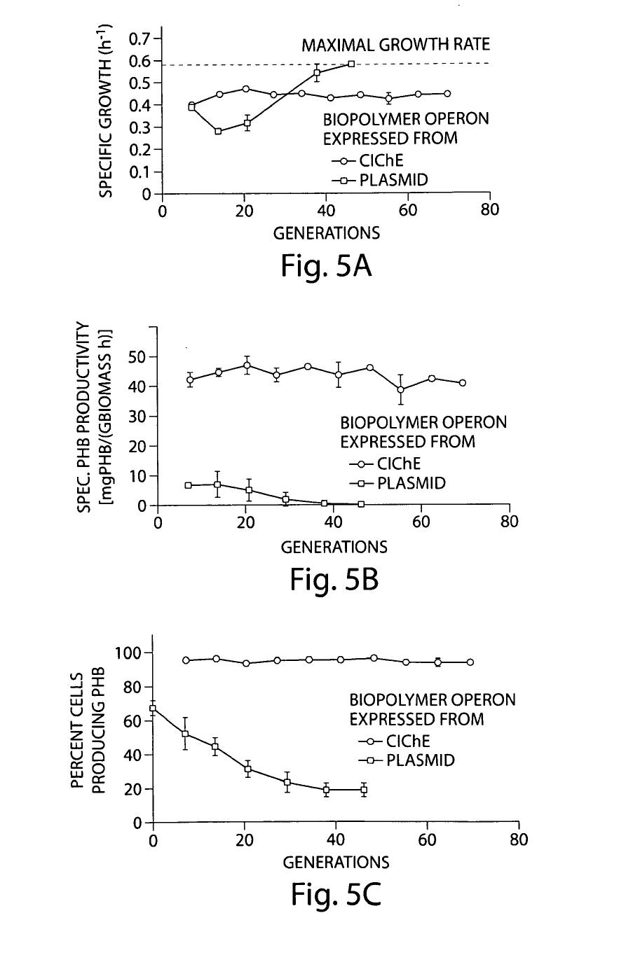

Next TGD was compared to conditions where allele segregation would take place. TGD (cat+PHB) recA− was maintained in exponential phase by subculturing for 70 generations without antibiotics and compared to the PHB plasmid-bearing strain, K12 recA− (pZE-tac-pha) with antibiotics. Unlike the prior experiment, the plasmid-bearing strain (but not the TGD strain) was grown in antibiotics, requiring cat to be active, but not necessarily the PHB operon. FIG. 5 shows the growth rates, specific PHB productivity, and percentage of cells producing PHB over time for the two strains. Without antibiotics, PHB productivity of the plasmid system went to zero in five doublings (data not shown). The plasmid system with antibiotics evolved to the maximal growth rate in 40 generations, which was accompanied by a loss of PHB productivity. The average specific growth rate of the TGD strain was 0.44 h⁻¹ compared to 0.58 h⁻¹ for the same strain without PHB production. This difference in growth rate would provide the conditions for a mutant population to overtake the culture, had it occurred. The TGD system maintained >90% of its PHB productivity for 70 generations. PHB plasmid-bearing cells underwent allele segregation, as PHB productivity was lost, but the selectable marker was maintained. The timescale of allele segregation observed compared favorably with our simple model of plasmid, considering an estimated 27 generations occurred before the plasmid-bearing strain began the allele segregation experiment, leading to a total of 67 generations to productivity loss. This shows that TGD constructs without selection pressure maintain recombinant expression under heavy metabolic burden for much longer than plasmids, even when antibiotics are used with the latter. We reiterate the no antibiotics were used for the TGD strain.

The ability to control gene dose is a useful feature for metabolic engineering and synthetic biology, because it allows precise control of gene expression. By varying the concentration of antibiotic used during the amplification stage, a range of copy numbers could be produced. FIG. 4 shows the effect of chloramphenicol on copy number in the constructs. Without the PHB operon, the copy number reaches a maximum of 45 copies with a linear range between 0-500 μg/mL chloramphenicol. When the PHB operon is included, there is an upward trend in copy number throughout the range of antibiotics used, however, it did not increase beyond ˜25 copies. A plausible explanation is that the PHB operon limits the copy number that is achievable due to the competition between a growth advantage brought about by the increased copies of the cat gene and a growth disadvantage caused by the metabolic burden imposed by the increased copies of the PHB operon. At low copy number, the increased growth advantage by additional copies of cat outweighs the disadvantage of more PHB operons. However, at higher copy number, the reverse becomes true, and the copy number reaches an equilibrium that is lower than if the PHB operon was not present.

TABLE 1
Oligonucleotides and their sequences

| Name | Description | Sequence (5′-3′) |
|---|---|---|
| Int P1 | chlb (s) | GCACCCTACGCATCGCCAGTTCTT |
| | | (SEQ ID NO: 1) |
| Int P2 | chlB (a) | CGCTCTCGGGCAAACTTTTCTGTGTT |
| | | (SEQ ID NO: 2) |
| Int P23 | Overlap primer for Anneal- | GGAACCTCTTACGTGCCGATCAAC |
| | Extend PCR | GGCCCCGTTGTCTTCACTGATCAACACT |
| | | (SEQ ID NO: 3) |
| Int P3 | cat (s) | CGTTGATCGGCACGTAAGAGGTTCC |
| | | (SEQ ID NO: 4) |
| Int P4 | cat (a) | CCTTAAAAAAATTACGCCCCGCCC |
| | | (SEQ ID NO: 5) |
| Lam P1(s) | BamHI used for pTGD | TATCGGATCCCCAGTTCTTTCAAAA |
| | construction | ACGTCCACGCC |
| | | (SEQ ID NO: 6) |
| Lam P4(a) | SacI SphI used for pTGD | GGGAGCTCAGCATGCCCTTAAAAA |
| | construction | AATTACGCCCCGCCC |
| | | (SEQ ID NO: 7) |
| Lam P7(s) | EcoRI MluI used for pTGD | GGAATTCAACGCGTCCAGTTCTTTC |
| | construction | AAAAACGTCCACGCC |
| | | (SEQ ID NO: 8) |
| Lam P8(a) | XbaI used for pTGD | CGTCTAGAGCCCCGTTGTCTTCACT |
| | construction | GATCAACACT |
| | | (SEQ ID NO: 9) |
| PHB (s) | SphI used for pTGD cloning | GCAAGCATGCAGCTTCCCAACCTT |
| | | ACCAGAGGGCG |
| | | (SEQ ID NO: 10) |
| PHB (a) | MluI used for pTGD cloning | GCACGCGTCGGCAGGTCAGCCCAT |
| | | ATGCAG |
| | | (SEQ ID NO: 11) |
| Cm qPCR (s) | qPCR to measure copy number | CGCCTGATGAATGCTCATCC |
| | of cat in genome or in | (SEQ ID NO: 12) |
| | plasmids | |
| Cm qPCR (a) | qPCR to measure copy number | AGGTTTTCACCGTAACACGC |
| | of cat in genome or in | (SEQ ID NO: 13) |
| | plasmids | |
| bioA qPCR | qPCR to normalize cat copy | GTGATGCCGAAATGGTTGCC |
| (s) | number to number of copies | (SEQ ID NO: 14) |
| | of genome | |
| bioA qPCR | qPCR to normalize cat copy | GCGGTCAGACGCTGCAACTG |
| (a) | number to number of copies | (SEQ ID NO: 15) |
| | of genome | |

### Example 2

### Batch Culture Comparison of TGD-Polyhydroxybutyrate (PHB) to Plasmid-Based PHB Expression

### Strains

Strains and plasmids used in this study are listed in Table 2. pAGL20, a modified pJOE7 kindly provided by Anthony Sinskey, contains the genes phaAB from R. eutropha, encoding the β-ketothiolase and the acetoacetyl coenzyme-A reductase, phaEC from Allochromatium vinosum, encoding the two-subunit PHB polymerase on a kanamycin resistant backbone¹². pZE21 is a ColE1 plasmid with kanamycin resistance and green fluorescent protein (gfp) driven by a P_{L}-tetO promoter¹³.

TABLE 2
Strains and plasmids

| Name | Description | Reference |
|---|---|---|
| Strains | | |
| XL1-Blue | Cloning/Expression Strain of | Stratagene (La Jolla, |
| K12 recA::kan | E. coli 30 tandem copies of | Calif.) (Tyo and |
| TGD (cat + PHB) | PHB biosynthetic operon from | Stephanopoulos, |
| | pAGL20 on E. coli genome | Submitted) |
| Plasmids | | |
| pAGL20 | PHB biosynthetic pathway on | (Lawrence, Choi |
| | modified pJOE7 | et al. 2005) |
| pZE21 | Medium copy plasmid (ColE1 | (Lutz and |
| | origin, kan^{R}) | Bujard 1997) |
| pZE-Cm | pZE21 with kan^{R} replaced | |
| | with Cm^{R} | |
| pZE-Cm-phaA | pZE derivative with cat and | |
| | R. eutrophus phaA | |
| pZE-Cm-phaB | pZE derivative with cat and | |
| | R. eutrophus phaB | |
| pZE-Cm-phaEC | pZE derivative with cat and | |
| | R. eutrophus phaA | |
| pZE-Cm-gfp | pZE derivative with cat and | |
| | gfp | |
| pZE-Cm-phaECAB | pZE derivative with cat and | |
| | pAGL20 PHB operon | |
| pZE-kan-tac-pha | pZE derivative with pAGL20 | |
| | PHB operon driven by strong | |
| | promoter (p_{tac}) | |

Cloning was performed using standard techniques and materials from New England Biosciences (Beverly, Mass.), and all cloning steps were performed in DH5α (Invitrogen). Table 3 lists all primers used for PCR. pZE21 was digested SacI/AatII and ligated to a chloramphenicol acetyl transferase PCR product from pAC184 bearing the same sites to create pZE-Cm. Promoterless PCR products of phaA, phaB, phaEC, and phaECAB, were generated from pAGL20 and cloned into the KpnI/MluI sites of pZE-Cm for the systematic overexpression study. These plasmids were co-transformed with pAGL20 into XL-1 Blue and characterized. Whole operon promoter replacement was accomplished by synthesizing the tac promoter from oligonucleotides and cloning into the AatII/EcoRI site of pZE21. A promoterless phaECAB PCR product from pAGL20 was then cloned into the KpnI/MluI sites. This plasmid was transformed into XL-1 Blue and characterized.

TABLE 3
Oligonucleotides

| | Restric- | |
|---|---|---|
| | tion | |
| Name | Site | Sequence (5′-3′) |
| Cm (s) | AatII | AAA [symbol] CCGTTGATCGGCACGTA |
| | | AGAGGTTCC (SEQ ID NO: 16) |
| Cm (a) | SacI | AA [symbol] CCTTAAAAAAATTACGC |
| | | CCCGCCC (SEQ ID NO: 17) |
| phaA (s) | KpnI | GG [symbol] GCATGACTGACGTTGTC |
| | | ATCGTATCCGC(SEQ ID NO: 18) |
| phaA (a) | MluI | CG [symbol] CGGAAAACCCCTTCCTT |
| | | ATTTGCG (SEQ ID NO: 19) |
| phaB (s) | KpnI | GG [symbol] GCATGACTCAGCGCATT |
| | | GCGTATGTGAC (SEQ ID NO: 20) |
| phaB (a) | MluI | CG [symbol] CCGACTGGTTGAACCAG |
| | | GCCG (SEQ ID NO: 21) |
| phaEC (s) | KpnI | GGGGTACCGACGGCAGAGAGACAATC |
| | | AAATCATG (SEQ ID NO: 22) |
| phaEC (a) | MluI | CGACGCGTATGGAAACGGGAGGGAAC |
| | | CTGC (SEQ ID NO: 23) |
| tac promoter | | GAGCTGTTGACAATTAATCATCGGCT |
| (s) | | CGTATAATGTGTGG |
| | | (SEQ ID NO: 24) |
| tac promoter | AatII/ | [symbol] CCACACATTATACGAGCCGAT |
| (a) | EcoRI | GATTAATTGTCAACAGCTC [symbol] |
| | | (SEQ ID NO: 25) |
| qPCR phaA | | CGTTGTCATCGTATCCGCCG |
| (s) | | (SEQ ID NO: 26) |
| qPCR phaA | | GACTTCGCTCACCTGCTCCG |
| (a) | | (SEQ ID NO: 27) |
| qPCR phaB | | GTGGTGTTCCGCAAGATGAC |
| (s) | | (SEQ ID NO: 28) |
| qPCR phaB | | CGTTCACCGACGAGATGTTG |
| (a) | | (SEQ ID NO: 29) |
| qPCR phaE | | GGAGCAGAGCCAGTATCAGG |
| (s) | | (SEQ ID NO: 30) |
| qPCR phaE | | CACCCTGGATGTAGGAGCCC |
| (a) | | (SEQ ID NO: 31) |

K12::PHBtac Cm20 ΔrecA—Tandem gene duplication strain expressing PHB operon. This strain is based on K12 and has used the TGD method to create tandem copies of a (PHB+chloramphenicol) DNA construct resistant to 1.7 mg/mL chloramphenicol. recA has been deleted by P1 phage transduction to stabilize the tandem repeats. No antibiotics were used in the PHB production fermentation.
XL1(pZE-tac-pha)+Cm—XL-1 Blue (Stratagene commercial expression strain) harboring plasmid pZE-tac-pha (described above). This would be a comparable plasmid-based PHB production strain used by other research groups. Cm+means 34 μg/mL chloramphenicol was used during PHB production fermentation to maintain the plasmid.
XL1(pZE-tac-pha)−Cm—As above, except no antibiotics were used in the PHB production fermentation.

### Conditions

Strains were grown as follows. Strains were cultured in the minimal media, MR with 20 g/L glucose at 37° C. (called MR from here forward). MR medium was prepared as defined previously¹⁴. Luria-Bertani (LB) broth was used for growth on solid media and standard preparations of cells.

Samples were taken throughout the course of growth and analyzed. Analytical procedures were as follows.

Cell densities were monitored at 600 nm using an Ultraspec 2100pro (Amersham Biosciences, Uppsala, Sweden). PHB concentrations were determined as follows using a fluorescence-based PHB measurement method. Cells were quantitatively stained with nile red to give a fluorescence signal proportional to PHB content¹⁶. Briefly, cells were resuspended in deionized water to an A730=0.4. 3 μL of a 1 mg/mL nile red (Sigma-Aldrich, St. Louis, Mo.), in dimethyl sulfoxide solution was used to stain intracellular PHB granules to fluoresce at 585 nm, and 1 uL of a 1 mM bis-(1,3-dibutylbarbituric acid)trimethine oxonol (oxonol) (Invitrogen, Carlsbad, Calif.) was used as a live/dead stain which fluoresces at 530 nm. Cells were incubated in the dark for 30 min at room temperature then sorted on a MoFlo fluorescence activated cell sorter (FACS) (Dako, Carpinteria, Calif.). Cells were sorted based on the PE filter set (for nile red fluorescence) and FITC filter set (for oxonol). Cells were collected that did not fluoresce for oxonol (living) and were in the top 0.1% of the nile red fluorescence distribution (high PHB).

Alternatively, chemical PHB analysis can be conducted as shown previously¹⁷.

For copy number measurement of phaB, the following 2 primers were used.

| phaB (s) | |
|---|---|
| GTGGTGTTCCGCAAGATGAC | (SEQ ID NO: 28) |
| phaB (a) | |
| CGTTCACCGACGAGATGTTG | (SEQ ID NO: 29) |

### Steady-State, Balanced Growth Accumulation

For measuring PHB specific productivity, the direct measurements of growth rate and PHB content must be taken while the cells are growing at steady state. Chemostats were not conducive to measuring many different strains in parallel. Instead, a protocol was developed to measure PHB at pseudo-steady state, or balanced, growth in shake flasks. To this end, we measured the PHB accumulation from early log phase through early stationary phase in a shake flask. Because it was difficult to have a repeatable lag phase, data was analyzed vs A₆₀₀ rather than vs time. PHB content is high as the cell begins to grow, due to accumulation from the stationary phase inoculum. As the initial effects are diluted away by growth, the PHB content falls. Finally, as the cells enter stationary phase, growth slows, and the PHB content rises again. Between A₆₀₀=2−3, the PHB is constant, and represent a regime where PHB accumulation is at steady state. To verify the PHB accumulation was at steady state, balanced growth, the strain were subcultured into pre-warmed shake flasks as the culture approached A₆₀₀=2, and the PHB content was measured over several subculturings (data not shown). PHB content did not change, confirming that the PHB accumulation was at steady state. The region between A₆₀₀=2-3 was chosen for further assays.

### Results

The above strains were cultured as described above and PHB production was measured as shown in Table 4. These results show that PHB specific productivity for the TGD strain is the same as the plasmid+antibiotics strain.

TABLE 4
Exponential growth parameters for PHB production

| | | Exp. | PHB Spec. |
|---|---|---|---|
| | | Phase | Prod. |
| Strain | μ (h⁻¹) | % PHB* | (g/(L − h))** |
| K12::PHBtac- | 0.52 +/− 0.05 | 11% | 0.064 |
| Cm20ΔrecA − Cm | | | |
| XL1 (pZE-tac-pha) + | 0.23 +/− 0.01 | 22% | 0.067 |
| Cm | | | |
| XL1 (pZE-tac-pha) − | 0.32 +/− 0.00 | 2% | 0.006 |
| Cm | | | |

*Exponential phase PHB production. Steady-state, balanced growth accumulation (as described above). Measured at A₆₀₀ = 2.0.
**PHB Specific Productivity - calculated from μ and Exp. Phase % PHB. This is the per cell flux to PHB.

Although TGD may be used more commonly in continuous processes, this data shows that TGD performs as well as plasmids in producing PHB in batch fermentation. TGD does much better than plasmids in continuous fermentation. It also shows that TGD can be active without antibiotics, while antibiotics are necessary for plasmids.

### Example 3

### Genetic Stability of TGD in Continuous Culture

Strain K12::PHBtac-Cm20ΔrecA-Cm (as described above) was grown in a nitrogen limited chemostat for ˜20 generations.

Nitrogen-Limited Chemostat PHB Production

PHB productivity measurements in chemostats allowed us to vary growth rates by controlling the dilution rate under aerobic, nitrogen-limiting conditions. The growth rate is constrained, but the relative expression of the PHB pathway is held constant.

Nitrogen-limited chemostat experiments were performed in a 3 L stirred glass vessel using the BioFlo 110 modular fermentation system (New Brunswick Scientific, Edison, N.J.) with a 1 L working volume. Bioreactor controllers were set to pH=6.9, adjusted by 6 N NaOH through controller, 30% dissolved oxygen, controlled by adjusting feed oxygen concentration, and temperature at 37° C., controlled by a thermal blanket and cooling coil. Gas flow was set at 3 L/min and agitation at 400 rpm. Antifoam SE-15 (Sigma-Aldrich, St. Louis, Mo.) was diluted in water and added by peristaltic pump to control foaming.

Sterile MR media without antibiotics was fed by peristaltic pump at flowrates of 50, 100, and 300 mL/h and culture broth was removed from the reactor using a level-stat. 10 mL samples were taken for characterization after four residence times at a given flowrate. Approach to steady state was verified by monitoring glucose concentration in reactor. Three measurements, each spaced one residence time apart, were taken at each steady state. Ammonium, the only nitrogen source, was monitored semi-quantitatively by NH₄⁺ test strips (Merck KGaA, Darmstadt, Germany), and was always below 10 mg/L (the lowest measurable value on the test strip). Chemostats were run in replicate, and glucose was in excess at all points except D=0.1 h⁻¹, where a dual nutrient limitation existed. Contamination was checked by streaking broth on nile red LB plates and checking that all colonies were PHB⁺ by nile red fluorescence.

Cell densities were monitored at 600 nm using an Ultraspec 2100pro (Amersham Biosciences, Uppsala, Sweden). Cell and PHB concentrations were determined as previously described above. Glucose concentrations were measured by a glucose analyzer (Yellow Springs Instruments, Yellow Springs, Ohio). Acetate was measured by high-pressure liquid chromatography using an Aminex HPX-87H ion-exclusion column (300 7.8 mm; Bio-Rad, Hercules, Calif.) and 14 mM H₂SO₄ mobile phase at 50° C. and 0.7 mL/min. Cell culture supernatant was kept at −20° C. before analysis.

### Results

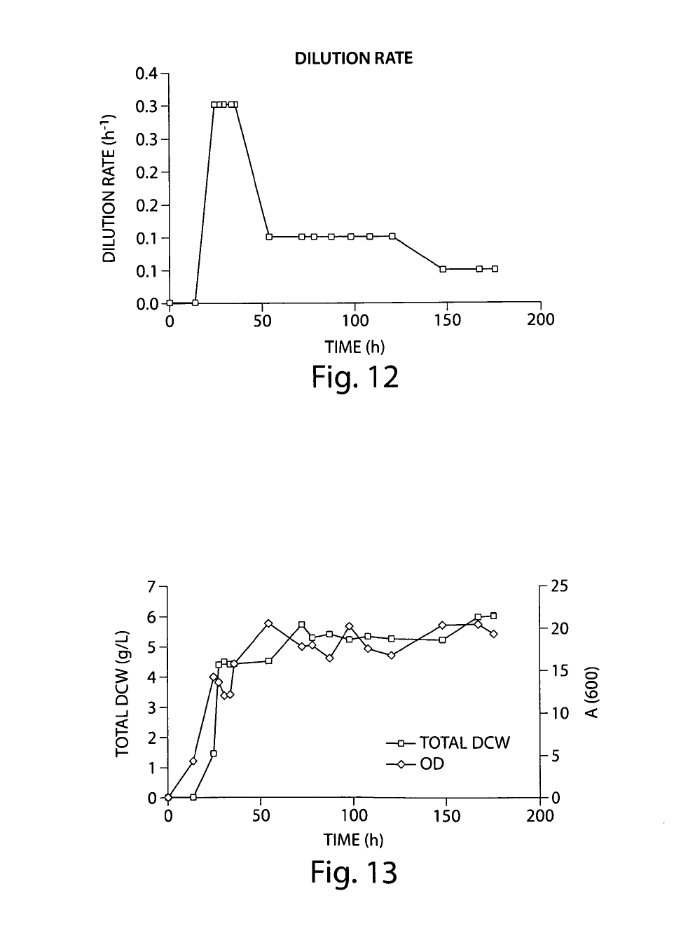

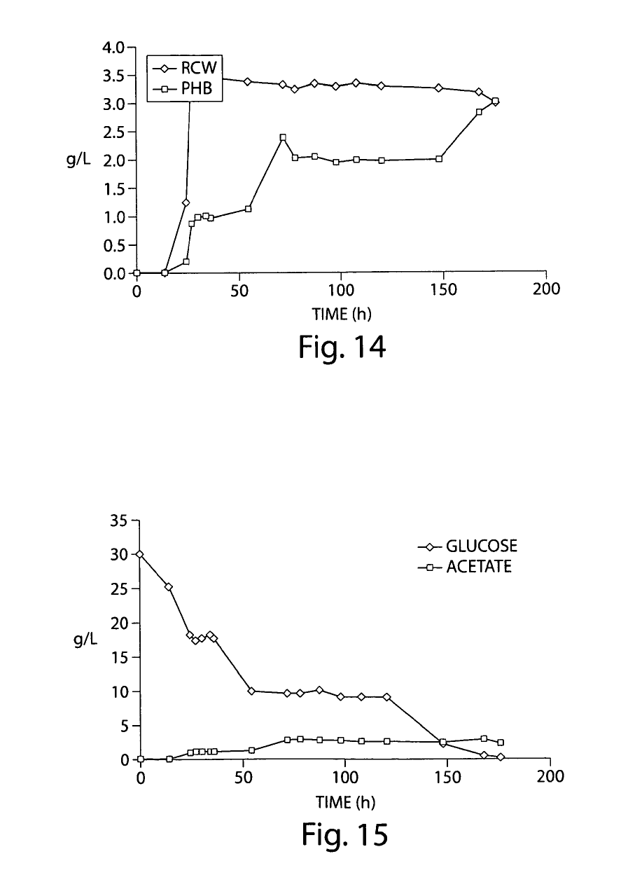

FIGS. 12-15 show the time course of the experiment, showing the TGD construct is genetically stable and expressed PHB over the ˜20 generations.

These plots provide evidence that over 175 hours the strain can maintain PHB productivity. This PHB productivity is maintained without any antibiotics, which is required for plasmids.

### REFERENCES FOR EXAMPLES 1-3

1. Firshein, W. & Kim, P. Plasmid replication and partition in Escherichia coli: is the cell membrane the key? Molecular Microbiology 23, 1-10 (1997).
2. Zhang, J. Evolution by gene duplication: an update. Trends in Ecology & Evolution 18, 292-298 (2003).
3. Olson, P. et al. High-Level Expression of Eukaryotic Polypeptides from Bacterial Chromosomes. Protein Expression and Purification 14, 160-166 (1998).
4. Xiaohai Wang, Z. W. N. A. D. S. G418 Selection and stability of cloned genes integrated at chromosomal. Biotech Bioeng 49, 45-51 (1996).
5. Andersson, D. I., Slechta, E. S. & Roth, J. R. Evidence That Gene Amplification Underlies Adaptive Mutability of the Bacterial lac Operon. Science 282, 1133-1135 (1998).
6. Boyd, D., Weiss, D. S., Chen, J. C. & Beckwith, J. Towards Single-Copy Gene Expression Systems Making Gene Cloning Physiologically Relevant: Lambda InCh, a Simple Escherichia coli Plasmid-Chromosome Shuttle System. J. Bacteriol. 182, 842-847 (2000).
7. Anderson, P. & Roth, J. Spontaneous Tandem Genetic Duplications in Salmonella typhimurium Arise by Unequal Recombination between rRNA (rrn) Cistrons. Proceedings of the National Academy of Sciences 78, 3113-3117 (1981).
8. Bentley, W. E. & Quiroga, O. E. Investigation of subpopulation heterogeneity and plasmid stability in recombinant Escherichia coli via a simple segregated model. Biotechnology and Bioengineering 42, 222-234 (1993).
9. Paulsson, J. & Ehrenberg, M. Noise in a minimal regulatory network: plasmid copy number control. Quarterly Reviews of Biophysics 34, 1-59 (2001).
10. Lee, S. Y., Lee, K. M., Chan, H. N. & Steinbüchel, A. Comparison of recombinant Escherichia coli strains for synthesis and accumulation of poly-(3-hydroxybutyric acid) and morphological changes. Biotechnology and Bioengineering 44, 1337-1347 (1994).
11. Kolisnychenko, V. et al. Engineering a Reduced Escherichia coli Genome. Genome Res. 12, 640-647 (2002).
12. Lawrence, A. G., Choi, J., Rha, C., Stubbe, J. & Sinskey, A. J. In Vitro Analysis of the Chain Termination Reaction in the Synthesis of Poly-(R)-beta-hydroxybutyrate by the Class III Synthase from Allochromatium vinosum. Biomacromolecules 6, 2113 -2119 (2005).
13. Lutz, R. & Bujard, H. Independent and tight regulation of transcriptional units in Escherichia coli via the LacR/O, the TetR/O and AraC/I1-12 regulatory elements. Nucl. Acids Res. 25, 1203-1210 (1997).
14. Wang, F. & Lee, S. Y. Production of poly(3-hydroxybutyrate) by fed-batch culture of filamentation-suppressed recombinant Escherichia coli. Appl. Environ. Microbiol. 63, 4765-4769 (1997).
15. Pont-Kingdon, G. in Methods in Molecular Biology: PCR Protocols, Vol. 226, Edn. Second Edition. (eds. J. M. S. Bartlett & D. Strirling) 511-515 (Humana Press Inc., Totowa, N.J.; 2003).
16. Tyo, K. E., Zhou, H. & Stephanopoulos, G. N. High-Throughput Screen for Poly-3-Hydroxybutyrate in Escherichia coli and Synechocystis sp. Strain PCC6803. Appl Environ Microbiol 72 (2006).
17. Taroncher-Oldenburg, G. & Stephanopoulos, G. Targeted, PCR-based gene disruption in cyanobacteria: inactivation of the polyhydroxyalkanoic acid synthase genes in Synechocystis sp. PCC6803. Appl Microbiol BiotechnoL 54, 677-680 (2000).

### Example 4

### Stabilized Gene Duplication Enables Long-Term Selection-Free Heterologous Pathway Expression

Engineering robust microbes for the biotech industry typically requires high-level, genetically stable expression of heterologous genes and pathways. Although plasmids have been used for this task, fundamental issues concerning their genetic stability have not been adequately addressed. Here we describe chemically inducible chromosomal evolution (CIChE), a plasmid-free, high gene copy expression system for engineering Escherichia coli. CIChE uses E. coli recA homologous recombination to evolve a chromosome with ˜40 consecutive copies of a recombinant pathway. Pathway copy number is stabilized by recA knockout, and the resulting engineered strain requires no selection markers and is unaffected by plasmid instabilities. Comparison of CIChE-engineered strains with equivalent plasmids revealed that CIChE improved genetic stability approximately tenfold and growth phase-specific productivity approximately fourfold for a strain producing the high metabolic burden—biopolymer poly-3-hydroxybutyrate. We also increased the yield of the nutraceutical lycopene by 60%. CIChE should be applicable in many organisms, as it only requires having targeted genomic integration methods and a recA homolog.

### Introduction

Recent breakthroughs in metabolic engineering have made it easier to overproduce biochemical products from renewable resources. Such advances include fabricating large synthetic pathways (de novo synthesized DNA sequences)^{1,2} and optimizing pathway expression through transcription- or translation-level engineering^{3,4}, which is essential to avoid buildup of toxic products. This progress has relied mainly on plasm id-based gene expression or single-copy genomic integration.

Although plasm ids are easy to insert into a cell and allow strong gene expression, they suffer from genetic instability due to three processes that reduce the number of active recombinant alleles in a culture⁵: (i) segregational instability, in which unequal distribution of plasmids to daughter cells results in plasmid-free cells; (ii) structural instability, in which some plasmids contain an altered DNA sequence that causes incorrect expression of the desired proteins; and (iii) allele segregation, in which productive plasmids are displaced by non-productive plasmids, leading to nonproductive cells that are resistant to selection pressure.

Whereas various strategies have been implemented to reduce segregational and structural instability⁶, allele segregation, which is not mitigated by selection markers or post-segregational killing mechanisms (PSK), has not been considered as a potential mechanism for productivity loss in biotechnology. Even so, it is likely that allele segregation has affected many pathway-engineering efforts by decreasing product yield and productivity of the desired chemical in batch or continuous fermentation. This unaddressed plasmid problem is ubiquitous in industrial biotechnology and will likely affect future endeavors to produce bioproducts using minimal-genome cells⁷.

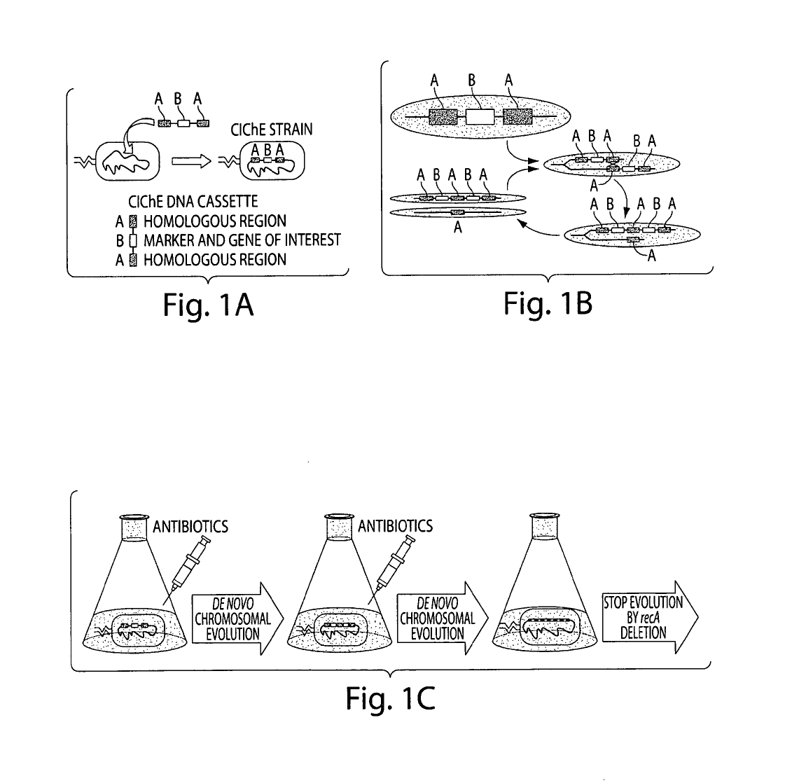

These three sources of instability suggest that the stable expression of genetic constructs through de novo chromosomal engineering, rather than artificial plasmid-based systems, is much needed to advance microbial overproduction using heterologous pathways. Here we present such a technique, CIChE, for biosynthetic pathway engineering in microbial hosts (FIG. 1) to circumvent allele segregation, a fundamental flaw in plasmid-based gene expression (FIG. 2). We use a mathematical model to explain that random plasmid inheritance, rather than mutation rates, drives productivity loss, whereas ordered inheritance, such as CIChE, can stabilize pathway productivity tenfold longer. We also demonstrate that CIChE allows cells with heavy metabolic burdens to remain productive and maintain or increase yields for many more generations than do analogous plasmid constructs. These results open the possibility for the broad use of CIChE-engineered microbes in large-scale industrial production.

### Results

### Random Distribution, Not Mutation Rates, Limit the Genetic Stability of Plasmids

Although structural and segregational instability have been understood for some time⁵, strategies devised to mitigate these instabilities, such as selection markers and post-segregational killing mechanisms, can maintain active plasmids for only ˜35 generations (an example of plasmid productivity loss in the presence of antibiotics is presented later). This loss in productivity is too rapid to be explained by mutation alone because mutations in all copies of the plasmid should not accumulate fast enough. If antibiotics were used to maintain selection pressure, this phenomenon could not be explained by structural or segregational instability.

Allele segregation, in contrast, can explain how a rare initial mutation can be rapidly propagated in a culture, decreasing productivity regardless of mutation rate. The logic is as follows: because recombinant pathways typically place heavy metabolic burdens on the cell and because the products can be cytotoxic, these pathways reduce cellular fitness. Therefore, displacement of productive plasmids with mutant nonproductive ones confers growth advantages that drive further propagation of such displacements. This is the phenomenon of allele segregation at work. Displacement occurs when plasmids are randomly distributed from mother to daughter cells. Such unbalanced distribution is reminiscent of segregational instability⁸. In that scenario, daughter cells that do not receive plasmids are removed by antibiotic selection. However, in the case of allele segregation, the nonproductive plasmids still confer antibiotic resistance because the mutation occurs in the recombinant pathway gene and not in the resistance gene.

FIG. 2(a) illustrates this process schematically. In one copy of a plasmid, a mutation occurs in the gene(s) of interest without affecting the selection marker. This is essentially a structural instability event. Next, the mutant plasmid is copied, and upon cell division, two outcomes are possible: either each daughter cell receives one copy of the mutant plasmid, or one daughter cell receives both mutant copies. In the latter case, the daughter cell receiving both mutant plasmids has effectively doubled its growth advantage and now produces less recombinant product. In subsequent generations, the cell with two mutant plasmids can give rise to additional nonproductive daughter cells by replacing unmutated plasmids while maintaining antibiotic resistance. This process is similar to the well-studied phenomenon of plasmid incompatibility, where two plasmid populations with similar origins-of-replication spontaneously segregate due to random partitioning and random plasmid replication initiation⁹⁻¹¹.

We constructed a population-balance model of plasmid propagation to compare the rates of productivity loss by allele segregation (by means of random inheritance and structural instability) to those resulting from genetic mutation alone (by means of ordered inheritance and structural instability) (See equations presented in Example 5). For a typical 40-copy plasmid, genetic mutation will inactivate one allele copy in ˜10 generations. With ordered inheritance, this leads to a slow loss of productivity over ˜500 generations (FIG. 2b). In contrast, allele segregation leads to productivity loss in only ˜40 generations (FIG. 2b). Phenomena not considered here, including random plasmid replication initiation and segregational instability, may increase further the rate of productivity loss^{8,11}. Thus, allele segregation results in drastic decreases in productivity. Importantly, mutation rates have little effect on longevity compared to the effects of random distribution of alleles. Hence, strategies to decrease mutation rates would be unlikely to improve plasmid productivity. On the other hand, a method that prevents the random distribution of plasmids from mother to daughter cells could substantially improve genetic stability, and thus facilitate long-term, selection-free, industrial-scale production of commodity chemicals by engineered microbes.

### Recombinant Pathway Expression Using CIChE

Genomic integration methods guarantee ordered inheritance, which prevents random distribution of alleles, and thereby increases recombinant genetic stability. Nonetheless, typically only one copy of a metabolic pathway can be delivered at a time^{12,13}. When many copies of a pathway are needed to reach desired expression levels, one-at-a-time integration methods become tedious and inadequate for pathway engineering needs. Likewise, low copy plasmids have been used for pathway engineering¹⁴, but they have limited expression strength and are still randomly distributed. CIChE is based on tandem gene duplication, a naturally occurring phenomenon that generates many head-to-tail repeats of a DNA sequence in the chromosome and has been used to amplify single genes (FIG. 6)¹⁵⁻²⁰. This process gives rise to many copies of an allele on one continuous piece of DNA that is not randomly inherited by daughter cells.

Although gene amplification has been achieved in a number of systems¹⁶⁻²⁰, gene copy numbers are inherently unstable, due to subsequent loss in copy number after selection pressures are removed. This has limited the usefulness of gene amplification for pathway engineering. In CIChE, gene copy number loss is prevented after gene amplification by deleting recA, the enzyme that mediates recombination, thereby fixing gene copy number and avoiding copy number instabilities inherent in previous gene amplification methods.

CIChE begins with the delivery of a duplication-ready expression cassette to the chromosome (FIG. 1a). We first delivered a DNA cassette containing gene(s) of interest and the gene encoding the antibiotic marker chloramphenicol acetyl transferase (cat), flanked on both sides by identical, noncoding 1 kb regions of foreign DNA that have low homology to the E. coli genome (see FIG. 8 for DNA construct details), into the chromosome using the λInCh genomic integration system²¹, although any integration strategy would suffice. The large identical regions served as homologous substrates for the crossover event (FIG. 1b and FIG. 6). By subculturing the strain in increasing concentrations of chloramphenicol, the chromosome will evolve to contain higher gene copy numbers by recA-dependent homologous recombination (FIG. 1c). At the desired gene copy number, recA is deleted to prevent subsequent homologous recombination that could reduce copy number.

This strategy was implemented, and gene copy number was measured by qPCR to assess changes in the chromosome. Strains with either of two constructs, one construct without a gene of interest and one containing an operon to produce the biopolymer poly-3-hydroxybutyrate (PHB), evolved to higher gene copy number in response to increasing antibiotic concentrations. Gene copy number of recA-deleted strains did not change, consistent with the recA dependence on homologous recombination (FIG. 3). The ability of recA to act as an ‘on-off’ switch for changing gene copy number represents an important control for chromosome evolution. Maximal gene copy numbers of 40 and 30 were attained under the highest antibiotic selection pressures for the construct without a gene of interest and the PHB operon, respectively (FIG. 4a).

### Application of CIChE to Recombinant PHB and Lycopene Pathway Engineering

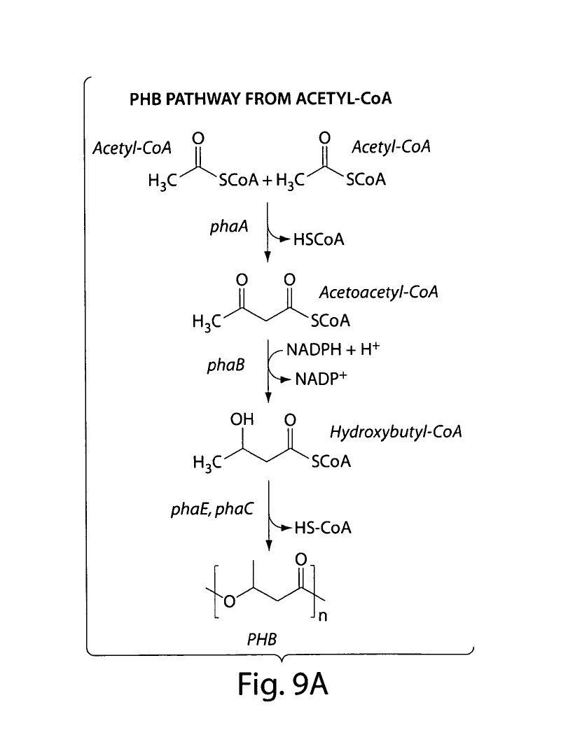

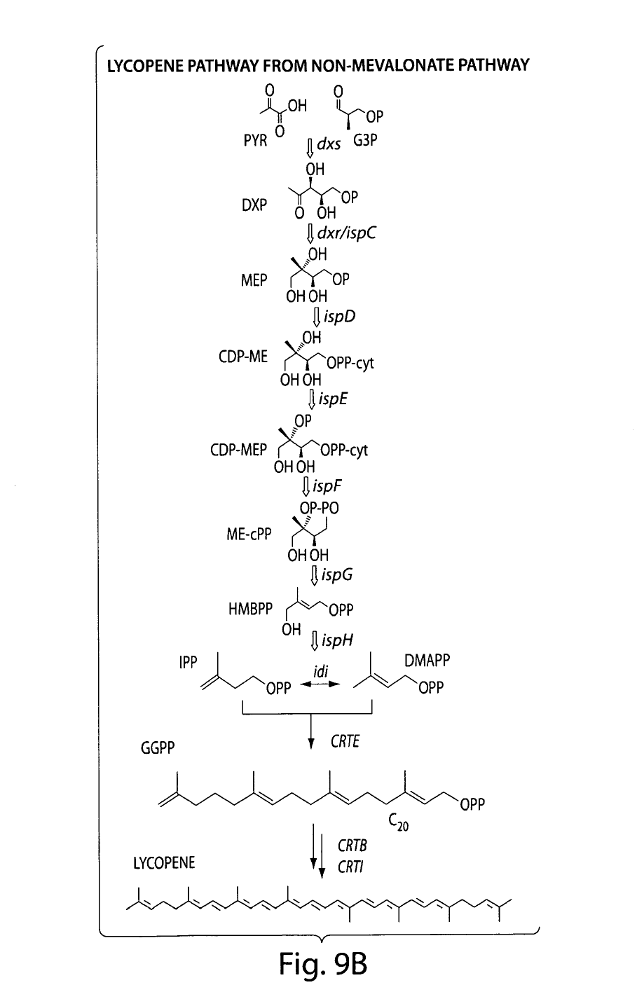

To demonstrate the practical application of CIChE and to compare this approach to engineering with plasm ids, we chose the expression of PHB and lycopene pathways (FIG. 9), both of which have been studied extensively using plasmid-based gene expression. PHB is a molecule with high metabolic burden, consuming large amounts of acetyl-CoA and NADPH for its synthesis, thereby reducing growth rates substantially²². Industrially, PHB is a member of a family of commercially interesting biodegradable biopolymers that are just now reaching commercial production²³. Likewise, isoprenoids, such as lycopene, have attracted considerable attention as a versatile class of pharmaceuticals and nutraceuticals²⁴. These two examples illustrate the versatility of CIChE for metabolic engineering of multi-gene pathways required for the production of biopolymers and small molecules.

CIChE-based yields compared favorably to plasmid yields for both PHB and lycopene in batch culture. pBR and p15A plasmids—which are maintained at 15-20 and 14-16 copies per cell, respectively, in stationary phase—were compared to equivalent CIChE constructs in the same gene copy number range, although plasmid copy number may fluctuate from growth to stationary phase^{25,26}. In cells with the most copies of CIChE-derived PHB operons, accumulated PHB was equivalent to that derived from plasmid-carrying cells, although anti-biotic selection was necessary for plasmid-based PHB production, but not for that of CIChE (FIG. 4b). Plasmid-carrying cells cultured without antibiotic selection produced 30% less PHB because the large metabolic burden quickly selected for cells that had reduced plasmid copy number (data not shown). PHB yields could also be tuned with the antibiotic strength used in the evolution process, which determined gene copy number.

Lycopene yields of CIChE constructs without antibiotic selection exceeded by 60% those found in equivalent plasmid strains with antibiotic selection in batch growth (FIG. 4c). Whereas prior metabolic engineering attempts have focused on upstream deregulation and pathway engineering to increase lycopene precursors²⁷⁻²⁹, here we show that considerable gains can be made by ensuring a high, constant pathway copy number.

Genetic stability, the most powerful benefit of CIChE, was tested by long-term subculturing while producing the metabolically demanding biopolymer PHB. Culture stability of the CIChE-PHB strain without antibiotics was compared to that of plasmids that were actively selected by antibiotics. Without antibiotics, plasmid-based PHB productivity drops to zero in five doublings (data not shown). The plasmid system with antibiotics lost PHB productivity after 40 generations (FIG. 5). For the plasmid-carrying strain, allele segregation could account for rapid loss of productivity, and the selection marker was maintained in the presence of antibiotics (FIG. 5). The observed time scale of allele segregation compared favorably with our plasmid subpopulation model, which predicted productivity loss would occur after ˜40 generations.

Productivity loss should be accompanied by increased growth rate, as was seen for the plasmid system (FIG. 5a). However, the average specific growth rate of the CIChE strain remained at 0.44 h−1 and did not evolve to 0.58 h−1, the specific growth rate for the same strain without PHB production (FIG. 5a). This difference in growth rate would provide excellent selection pressure for mutant, nonproductive cells to overtake the productive CIChE cells, but this did not occur, as growth rate remained constant (FIG. 5a).

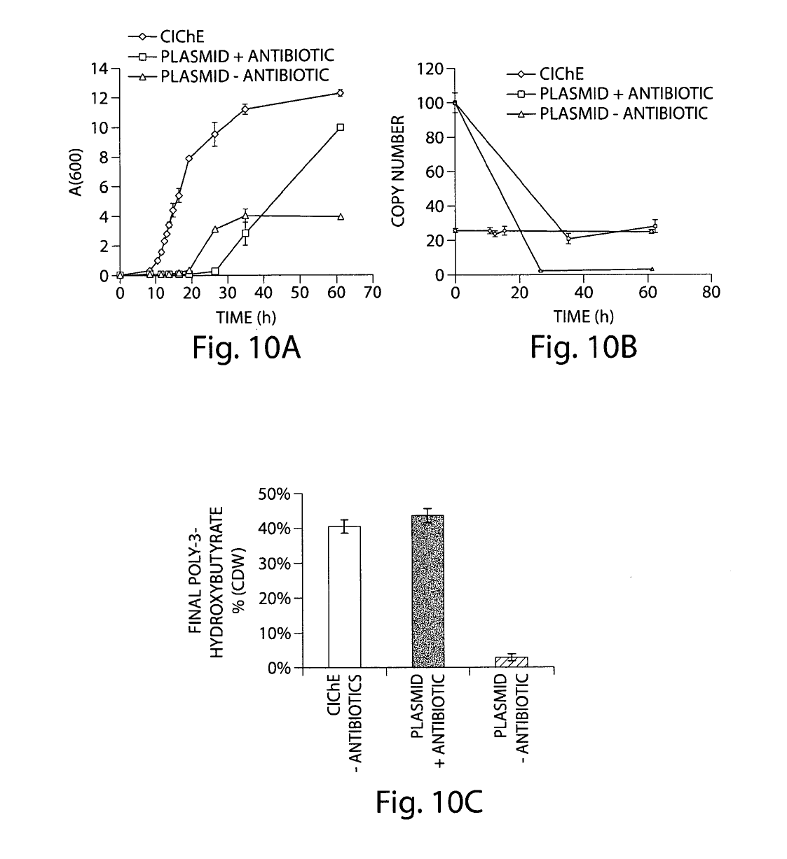

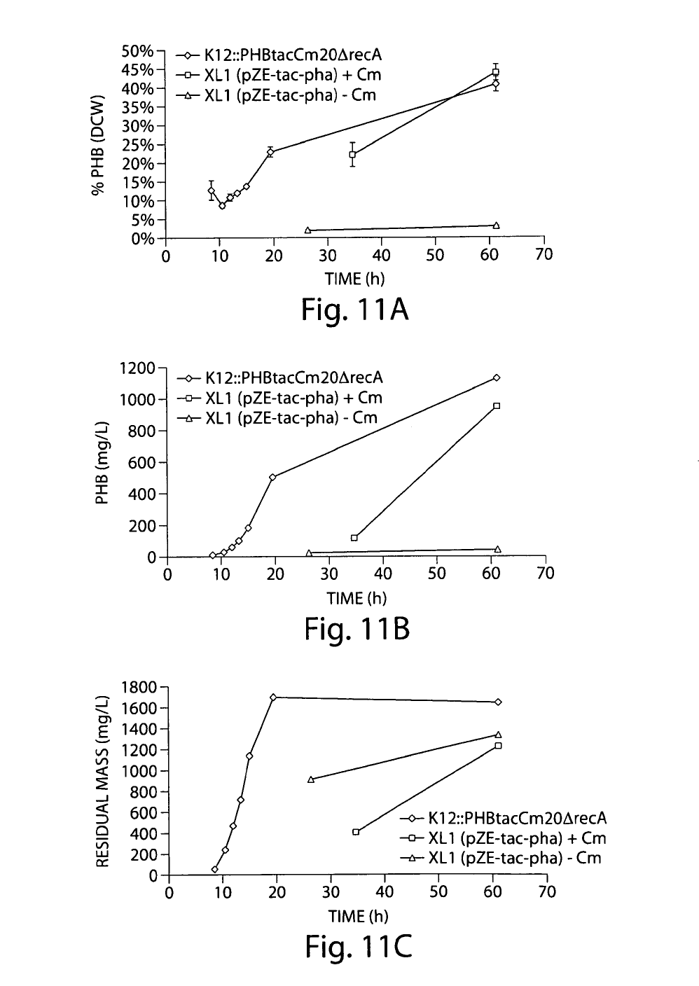

Furthermore, the CIChE strain maintained >90% of its PHB productivity for 70 generations. Notably, the specific productivity, or productivity on a per biomass basis, was also higher, a result of PHB operon copy number being unaffected by growth rate in the CIChE strain, whereas plasmids experience a downward pressure on copy number during growth, resulting in lower productivity⁸. The effect of growth on plasmid gene copy number is directly measured in FIG. 10. This shows that CIChE constructs without selection pressure maintain recombinant expression under heavy metabolic burden for many more generations than plasmids, even when antibiotics are used with the latter. No antibiotics were used in the production phase for the CIChE strain.

### Discussion

The role of allele segregation in limiting long-term gene expression and inducing precipitous plasmid productivity loss has long been overlooked. This phenomenon is likely to occur regularly, despite using antibiotic selection and stable cloning strains, particularly in subculturing experiments or chemostats, where productive plasmids are associated with a fitness penalty. Avoiding plasmid loss and reducing genetic mutation rates do not address the underlying mechanism of allele segregation, and are thus likely to be ineffective.

Although minimal genome approaches promise to decrease mutation frequencies by removing transposons, prophage and other genetic loci⁷, if plasmids are used to express key pathways, these approaches will likely fail. Our results suggest that random inheritance, not mutation rates, control longevity in plasmid systems. Inducible metabolic pathways can reduce the fitness penalty associated with productive alleles during growth, but this would not apply for growth-phase products or continuous fermentations⁶. Additionally, induction systems can be difficult to implement on industrial scales. Methods that force ordered inheritance will be much more stable and should underpin industrial biotechnology applications.

De novo chromosomally evolved pathways and genes, where new evolution of gene copy number can be directly controlled, are advantageous for high copy expression in many respects. Most importantly, CIChE avoids allele segregation, thereby considerably delaying overtake of cultures by mutant alleles that plague plasmid systems. This is achieved by linking all copies on a single strand of DNA in the chromosome and thus forcing ordered inheritance. As CIChE pathway—engineered cells can be propagated without antibiotics and do not relyon auxotrophic markers or post-segregational killing mechanisms, they offer a favorable alternative to selection-marker systems. A key feature of this method is the use of recA as an on-off switch to control the change in gene copy number of the recombinant genes. Without recA deletion, gene copy number in CIChE strains would decrease over time, as seen with other gene amplification strategies^{16,17,19}.

CIChE may also be useful for synthetic biology and metabolic engineering. CIChE constructed pathways and genes reach high gene doses comparable to those achievable using multicopy plasmids, unlike other genomic integration approaches that are tedious to extend to beyond five copies³⁰. We verified high gene dosage in CIChE cells by measuring gene copy number (FIG. 4a) and by achieving high PHB productivity and lycopene yield.

The copy number of the tandem genes is fixed in a population because the genes are integrated in the chromosome and homologous recombination is prevented by recA deletion. In contrast, plasmid copy numbers vary widely from cell to cell in a culture at a given moment⁸ and can change substantially between growth and stationary phases (FIG. 10)³¹. Effects that suppress plasmid copy number decrease yields, as is seen for lycopene (FIG. 4c) and PHB productivity (FIG. 5b). We were able to achieve PHB accumulation up to 70%

PHB dry cell weight (DCW) without selection pressure, equivalent to values obtained using plasmids where active selection is required³². Lee et al. were able to get only 22% PHB (DCW) with K12 E. coli using a parB partitioning plasmid³². We hypothesize that plasmid replication problems limited PHB production in K12, which we overcame using CIChE, boosting PHB threefold. The CIChE-lycopene system yielded up to ˜11,000 p.p.m., equivalent to an extensively engineered triple-gene knockout (gdhA/aceE/fdhF) strain²⁸. In the CIChE-lycopene strain the high yield relied on a high, fixed gene copy number, implying copy number may be as important as manipulation of metabolic networks for cellular engineering. Constant gene copy number will be valuable in synthetic control circuits, where transient variation in copy number could be problematic, leading to unintended responses and artifacts.

Furthermore, the gene copy number in CIChE can be tuned over a continuous range by the strength of antibiotic used in the gene-amplification step (FIG. 4a). By this, finely tuned expression levels can be generated, which may be useful for balancing metabolic pathways and in precisely designed synthetic control circuits. Plasmids are limited to low-, medium- or high-copy numbers and require promoter engineering to allow fine gradations in expression level^{6,33}.

CIChE has many potential biotech applications. CIChE constructs could be particularly useful in the production of low value—added commodity chemicals because it enables high-level, long-term expression of a multistep pathway. Such expression is required in continuous processes, which can have inherent economic advantages over batch processes, particularly for low-value products such as biofuels. As CIChE requires no antibiotics or auxotrophic markers during the production phase, it may enable use of low-cost feed stocks, such as corn stover, which are complex mixtures where auxotrophic selection can be ineffective³⁴. CIChE improves production when large growth penalties are associated with the metabolic pathway. This is likely for highly productive cells because large amounts of substrate are diverted away from biomass formation, and high concentrations of intermediates and products may be toxic to the cell. Because CIChE deters the establishment of fast-growing, low-productivity mutants, CIChE constructs are more tolerant than plasmid to growth penalties.

CIChE should be applicable in most industrially relevant host organisms. The only requirements are targeted genomic integration methods for the delivery of the CIChE cassette and for recombination knockout, and a recA homolog that can turn homologous recombination on and off. Because these requirements are widely met in gram-positive and gram-negative bacteria, yeast and other fungi, and, to a limited extent, mammalian cell lines, this method may be broadly useful for industrial recombinant expression because of the stability, high expression and lack of need for plasmid maintenance.

### Methods for Example 4

### Strains, Media and Oligonucleotides

E. coli K12 was used for the host strain of all CIChE and plasmid comparison experiments. DH5α (Invitrogen, San Diego, Calif.) was used for cloning steps. Lysogens and transfer strains, described in the λInCh protocol, were kindly provided by Dana Boyd and Jon Beckwith²¹. BW26,547 (recA::kan Lambda recA+), generated by Barry Wanner, used for PI phage transduction of the recA deletion allele to the CIChE strains, was received from Bob Sauer. Genomic DNA was isolated from Synechocystis PCC6803 using the Wizard Genomic Purification kit (Promega, Madison, Wis.). pAGL20, a modification of pJOE35 is a gift from Anthony Sinskey, containing the genes phaAB from Ralstonia eutropha, encoding the β-ketothiolase and the acetoacetyl coenzyme A reductase, and phaEC from Allochromatium vinosum, encoding the two-subunit PHB polymerase. pZE-tac-pha was generated from pZE21³⁶, with the PHB operon from pAGL20 and a Ptac promoter. pAC-LYC is based on published work³⁷. Luria-Bertani (LB) broth was used for routine culturing and for CIChE amplification. PHB production was measured when grown in minimal MR medium³⁸ with 20 g/l glucose. All oligonucleotides used in this study are listed in Tables 1, 3 and 4.

### Construction of CIChE Strain

pTrcHis2B (Invitrogen, San Diego, Calif.) was digested by SphI & ClaI, blunted by mung bean nuclease and ligated to yield only the pBR322 origin and β-lactamase. A 1 kb region of chlB from Synechocystis PCC6803 was PCR amplified using Int P1 and Int P2 primers. Likewise, cat from pAC-LYC37 was PCR amplified using Int P3 and Int P4 primers. Overlap PCR39 was used to connect the chlB and cat PCR products. The resulting DNA was PCR amplified by the Lam P1(s) and Lam P4(a) primers to introduce BamHI and SacI/SphI sites. The chlB/cat fragment was cloned into the truncated pTrcHis2B by BamHI/SacI sites. Another copy of chlB was PCR amplified by Lam P7(s) and Lam P8(a) primers to introduce EcoRI/MluI and XbaI sites for cloning. The second chlB fragment was cloned into the vector by EcoRI/XbaI sites resulting in the plasmid pTGD. This plasmid, pTGD, was used as a general integration platform (plasmid map in FIG. 8). pTGD-PHB was created by PCR amplification of the phaECAB operon with tac promoter and optimal RBS from pZE-tac-pha with PHB(s) and PHB(a) primers and cloned into the SphI/MluI sites of pTGD. Similarly, pTGD-LYC plasmid was constructed by PCR amplification of the CrtEBI operon from the pAC-LYC37 plasmid with CrtEBI(s) and CrtEBI(a) primers and cloned into the KpnI/MluI sites of pTGD. The resulting DNA constructs were transferred from the plasmids to the chromosome of K12 E. coli using the XInCh protocol²¹. This protocol delivers the plasmid construct to the chromosome, followed by a recombination step that removes the λ phage DNA from the chromosome.

Amplification of the construct was accomplished by subculturing the resulting strains in 5 ml LB medium with increasing chloramphenicol in 14 ml culture tubes. The CIChE strain was grown to stationary phase in 13.6 μg/ml chloramphenicol. 50 μl of culture was subcultured into a new culture tube (100× dilution). In the new tube, the chloramphenicol concentration was doubled from 13.6 μg/ml to 27.2 μg/ml and allowed to grow to stationary phase. This was repeated until the desired concentration (as high as 1,360 μg/ml) was reached. recA deletion was accomplished by P1 phage transduction of the recA::kan allele from BW26,547 to the CIChE strain following the Sauer P1 protocol (http://openwetware.org/wiki/Sauer:P1vir_phage_transduction). recA was routinely validated by UV sensitivity to 3,000 μJ energy using a Stratalinker UV crosslinker (Stratagene). Two colonies were chosen from the phage transduction at each antibiotic concentration for further analysis.

### Exponential Phase Genetic Stability Assay

K12 recA- (pZE-tacpha) and K12 recA-CIChE-PHB strain selected on 1,360 μg/ml chloramphenicol were cultured by continuous subculture, where the cells were transferred to a new flask shortly before entering stationary phase, thereby maintaining constant growth. Cells were grown in 250 ml Erlenmeyer shake flasks with 50 ml MR media at 37° C. and 225 r.p.m. Cells were inoculated at A₆₀₀=0.015 and were allowed to grow to late exponential phase (typically A₆₀₀=2.0). Cells were subcultured by inoculating the culture to A₆₀₀=0.015 in a prewarmed shake flask as above. By this, cells were continuously growing at maximal growth rate and did not enter stationary phase during the course of the experiment. Specific growth rate was estimated in each subculture based on the A₆₀₀ data points taken. PHB accumulation was measured as below and used to calculate PHB productivity as the product of specific growth rate and PHB accumulation, an approximation that holds at steady state.

qPCR Measurement of Gene Copy Number

Gene copy numbers were detected by qPCR on genomic DNA isolated from the appropriate strains using the Wizard Genomic Purification kit (Promega, Madison, Wis.). qPCR was carried out on a Bio-Rad iCycler using the iQ SYBR Green Supermix (Biorad, San Francisco, Calif.). cat copy numbers were detected and compared to the copy number of bioA, a nearby native gene in the chromosome. Tables 1, 3 and 4 have primers used for qPCR. Genomic DNA containing only one copy of the construct was diluted and used as a standard curve.

Poly-3-hydroxybutyrate Assay

End-point PHB accumulation of the strains was determined by inoculating an overnight culture of LB (1% vol/vol) into a 250 ml shake-flask with 50 ml of MR media. Cells were cultured at 37° C. for 72 h at 225 r.p.m. Cells were harvested by centrifugation, and PHB content was analyzed by hydrolysis to crotonic acid followed by high-performance liquid chromatography analysis as described elsewhere40. An authentic PHB standard was purchased from Sigma-Aldrich, St. Louis, Mo.

### Construction of Lycopene-Producing Strains and Lycopene Production

Overexpressions of dxs, idi and ispFD genes were performed by cloning the genes as an operon under a Trc promoter, which was constructed on pCL1920 plasmid back bone (pCL1920TrcMEP) with spectinomycin marker. dxs-idi-ispDF operon was initially constructed by cloning each gene from the genome of E. coli K12 MG1655 using the primers dxs(s), dxs(a), idi(s), idi(a), ispDF(s) and ispDFI(a) under pET21C+ plasmid with T7 promoter. Using the primers dxsidiispDF(s) and dxsidiispDF (a), dxs-idi-ispDF operon was sub-cloned into a NcoI- and KpnI-digested pTrcHis2B plasmid (Invitrogen, San Diego, Calif.) to create pTrcMEP. pTrcMEP plasmid was digested with BstZ17I and ScaI and cloned into PvuII-digested pCL1920 plasmid to construct the pC11920TrcMEP plasmid. The lycopene-producing CIChE strains were prepared by transferring the dxs-idi-ispDF operon plasmid pCL1920TrcMEP to CIChE-LYC strains selected on either 136 or 1,360 μg/ml chloramphenicol. The lycopene producing plasmid-based strain was constructed by cotransforming the plasmids pCL1920TrcMEP and pAC-LYC into K12 MG1655 recA background for comparison with the lycopene-producing CIChE strains.

To test the lycopene production from different strains, 50-ml cultures were started in a 250-ml flask with a 1% inoculation from an overnight 5-ml culture in LB medium. The strains were grown at 30° C. with 250 r.p.m. orbital shaking in R-glycerol minimal medium pH 7.0 (KH₂PO₄, 13.3 g/l; (NH₄)₂HPO₄, 4 g/l; citric acid, 1.7 g/l; EDTA, 0.0084 g/l; CoCl₂, 0.0025 g/l; MnCl₂, 0.015 g/l; CuCl₂, 0.0015 g/l; H₃BO₃, 0.003 g/l; Na₂MoO₄, 0.0025 g/l; Zn(CH₃COO)₂, 0.008 g/l; Fe(III) citrate, 0.06 g/l; thiamine, 0.0045 g/l; MgSO₄, 1.3 g/l; glycerol 15 g/l). Induction with isopropyl-b-D-thiogalactopyranoside was used to induce the dxs-idi-ispDF pathway. Surfactant Tween 80, was added at 0.5% (wt/vol) as required. Lycopene production culture was maintained for 48 h. Five colonies from the solid media was used to get the s.d.

To determine the lycopene content of the cells, 100 μl of E. coli cells was harvested by centrifugation at 18,000 g for 3 min. The cell pellet was washed and then extracted in 1 ml of acetone at 55° C. for 15 min with intermittent vortexing. The lycopene content in the supernatant was quantified through absorbance at 475 nm, and concentrations were calculated through a standard curve and appropriate dilution factor. The entire extraction process was performed in reduced light conditions to prevent photobleaching and degradation. Cell mass was calculated by correlating dry cell with OD₆₀₀ for use in parts per million (mg lycopene/g dry cell weight) calculations.

### Fluorescence-Activated Cell Sorting (FACS) Assay

Percentage of cells producing PHB was determined by FACS using Nile red to stain the PHB granules⁴⁰. Samples were collected from the exponential phase subculturing experiment, and allowed to grow to late stationary phase (to maximize the PHB in a cell). 200 μl of cell culture was mixed with 800 μl isopropanol to fix the cells. This was incubated for 10 min and then resuspended in 10 mg/ml MgCl₂ with 3 μg/ml Nile red. Cells were stained with Nile red for 30 min in the dark and analyzed on a Becton Dickinson FACScan. Cells were scored as containing PHB if the fluorescence was above the 95^{th} percentile of a distribution of cells containing no PHB.

TABLE 4
Oligonucleotides

| Name | Description | Sequence (5′-3′) |
|---|---|---|
| CrtEBI(s) | KpnI used for pTGD cloning | CGGGGTACCGCCAGTCACTATGGCGTGCTG |
| | | CTAGCGC (SEQ ID NO: 32) |
| CrtEBI(a) | MluI used for pTGD cloning | CGACGCGTGGCCGCCCGCCTAAACGGGACG |
| | | C (SEQ ID NO: 33) |
| dxs(s) | NdeI used for pET-dxs cloning | CGGCATATGAGTTTTGATATTGCCAAATAC |
| | | CCG (SEQ ID NO: 34) |
| dxs(a) | NheI used for pET-dxs cloning | CGCGGCTAGCTTATGCCAGCCAGGCCTTGA |
| | | TTTTG (SEQ ID NO: 35) |
| idi(s) | Nhel used for pET-dxsidi | CGCGGCTAGCGAAGGAGATATACATATGCA |
| | cloning | AACGGAACACGTCATTTTATTG |
| | | (SEQ ID NO: 36) |
| idi(a) | EcoRI used for pET-dxsidi | CGCGGAATTCGCTCACAACCCCGGCAAATG |
| | cloning | TCGG (SEQ ID NO: 37) |
| ispDF(s) | EcoRI used for pET-dxsidiispDF | CGGCGAATTCGAAGGAGATATACATATGGC |
| | cloning | AACCACTCATTTGGATGTTTG |
| | | (SEQ ID NO: 38) |
| ispDF(a) | XhoI used for pET-dxsidiispDF | GCGCTCGAGTCATTTTGTTGCCTTAATGAG |
| | | TAGCGCC (SEQ ID NO: 39) |
| dxsidiispDF(s) | NcoI used for pTrc-dxsidiispDF | TAAACCATGGGTTTTGATATTGCCAAATAC |
| | | CCG (SEQ ID NO: 40) |
| dxsidiispDF(a) | KpnI used for pTrc-dxs-idi-ispDF | CGGGGTACCTCATTTTGTTGCCTTAATGAG |
| | | TAGCGC (SEQ ID NO: 41) |

### REFERENCES FOR EXAMPLE 4

1. Gibson, D. G. et al. Complete chemical synthesis, assembly, and cloning of a Mycoplasma genitalium genome. Science 319, 1215-1220 (2008).
2. Kodumal, S. J. et al. Total synthesis of long DNA sequences: Synthesis of a contiguous 32-kb polyketide synthase gene cluster. Proc. Natl. Acad. Sci. USA 101, 15573-15578 (2004).
3. Pfleger, B. F., Pitera, D. J., Smolke, C. D. & Keasling, J. D. Combinatorial engineering of intergenic regions in operons tunes expression of multiple genes. Nat. Biotechnol. 24, 1027-1032 (2006).
4. Win, M. N. & Smolke, C. D. A modular and extensible RNA-based gene-regulatory platform for engineering cellular function. Proc. Natl. Acad. Sci. USA 104, 14283-14288 (2007).
5. Friehs, K. Plasmid copy number and plasmid stability. in New Trends and Developments in Biochemical Engineering vol. 86 (ed. Scheper, T. H.) 47-82, (Springer Berlin, Heidelberg, Germany, 2004).
6. Keasling, J. D. Gene-expression tools for the metabolic engineering of bacteria. Trends Biotechnol. 17, 452-460 (1999).
7. Kolisnychenko, V. et al. Engineering a reduced Escherichia coli genome. Genome Res. 12, 640-647 (2002).
8. Bentley, W. E. & Quiroga, O. E. Investigation of subpopulation heterogeneity and plasmid stability in recombinant Escherichia coli via a simple segregated model. Biotechnol. Bioeng. 42, 222-234 (1993).
9. Ishii, K., Hashimoto-Gotoh, T. & Matsubara, K. Random replication and random assortment model for plasmid incompatibility in bacteria. Plasmid 1, 435-445 (1978).
10. Novick, R. P. Plasmid incompatibility. Microbiol. Rev. 51, 381-395 (1987).
11. Novick, R. P. & Hoppensteadt, F. C. On plasmid incompatibility. Plasmid 1, 421-434 (1978).
12. Snell, K. D., Draths, K. M. & Frost, J. W. Synthetic modification of the Escherichia coli chromosome: enhancing the biocatalytic conversion of glucose into aromatic chemicals. J. Am. Chem. Soc. 118, 5605-5614 (1996).
13. Wang, Y. & Pfeifer, B. A. 6-deoxyerythronolide B production through chromosomal localization of the deoxyerythronolide B synthase genes in E. coli. Metab. Eng. 10, 33-38 (2008).
14. Martin, V. J., Pitera, D. J., Withers, S. T., Newman, J. D. & Keasling, J. D. Engineering a mevalonate pathway in Escherichia coli for production of terpenoids. Nat. Biotechnol. 21, 796-802 (2003).
15. Zhang, J. Evolution by gene duplication: an update. Trends Ecol. Evol. 18, 292-298 (2003).
16. Olson, P. et al. High-level expression of eukaryotic polypeptides from bacterial chromosomes. Protein Expr. Purif. 14, 160-166 (1998).
17. Wang, X., Wang, Z. & Da Silva, N. A. G418 Selection and stability of cloned genes integrated at chromosomal delta sequences of Saccharomyces cerevisiae. Biotechnol. Bioeng. 49, 45-51 (1996).
18. Borth, N., Zeyda, M., Kunert, R. & Katinger, H. Efficient selection of high-producing subclones during gene amplification of recombinant Chinese hamster ovary cells by flow cytometry and cell sorting. Biotechnol. Bioeng. 71, 266-273 (2000).
19. Kim, N. S., Kim, S. J. & Lee, G. M. Clonal variability within dihydrofolate reductase-mediated gene amplified Chinese hamster ovary cells: stability in the absence of selective pressure. Biotechnol. Bioeng. 60, 679-688 (1998).
20. Nakanishi, F. et al. Evaluation of stability in the DHFR gene amplification system using fluorescence in situ hybridization. in Animal Cell Technology: Basic & Applied Aspects vol. 10 (eds. Kitagawa, Y., Matsuda, T. & Iijima, S.) 25-263 (Kluwer Academic Publishers, Dordrecht, The Netherlands, 1999).
21. Boyd, D., Weiss, D. S., Chen, J. C. & Beckwith, J. Towards single-copy gene expression systems making gene cloning physiologically relevant: lambda InCh, a simple Escherichia coli plasmid-chromosome shuttle system. J. Bacteriol. 182, 842-847 (2000).
22. Hong, S. H., Park, S. J., Moon, S. Y., Park, J. P. & Lee, S. Y. In silico prediction and validation of the importance of the Entner-Doudoroff pathway in poly(3-hydroxybutyrate) production by metabolically engineered Escherichia coli. Biotechnol. Bioeng. 83, 854-863 (2003).
23. Madison, L. L. & Huisman, G. W. Metabolic engineering of poly(3-hydroxyalkanoates): from DNA to plastic. Microbiol. Mol. Biol. Rev. 63, 21-53 (1999).
24. Klein-Marcuschamer, D., Ajikumar, P. K. & Stephanopoulos, G. Engineering microbial cell factories for biosynthesis of isoprenoid molecules: beyond lycopene. Trends Biotechnol. 25, 417-424 (2007).
25. Hiszczynska-Sawicka, E. & Kur, J. Effect of Escherichia coli IHF mutations on plasmid p15A copy number. Plasmid 38, 174-179 (1997).
26. Ivanov, I. G. & Bachvarov, D. R. Determination of plasmid copy number by the boiling method. Anal. Biochem. 165, 137-141 (1987).
27. Alper, H., Jin, Y.-S., Moxley, J. F. & Stephanopoulos, G. Identifying gene targets for the metabolic engineering of lycopene biosynthesis in Escherichia coli. Metab. Eng. 7, 155-164 (2005).
28. Alper, H., Miyaoku, K. & Stephanopoulos, G. Construction of lycopene-overproducing Escherichia coli strains by combining systematic and combinatorial gene knockout targets. Nat. Biotechnol. 23, 612-616 (2005).
29. Farmer, W. R. & Liao, J. C. Improving lycopene production in Escherichia coli by engineering metabolic control. Nat. Biotechnol. 18, 533-537 (2000).
30. Datsenko, K. A. & Wanner, B. L. One-step inactivation of chromosomal genes in Escherichia coli K-12 using PCR products. Proc. Natl. Acad. Sci. USA 97, 6640-6645 (2000).
31. Paulsson, J. & Ehrenberg, M. Noise in a minimal regulatory network: plasmid copy number control. Q. Rev. Biophys. 34, 1-59 (2001).
32. Lee, S. Y., Lee, K. M., Chan, H. N. & Steinbüchel, A. Comparison of recombinant Escherichia coli strains for synthesis and accumulation of poly-(3-hydroxybutyric acid) and morphological changes. Biotechnol. Bioeng. 44, 1337-1347 (1994).
33. Alper, H., Fischer, C., Nevoigt, E. & Stephanopoulos, G. Tuning genetic control through promoter engineering. Proc. Natl. Acad. Sci. USA 102, 12678-12683 (2005).
34. Pronk, J. T. Auxotrophic yeast strains in fundamental and applied research. Appl. Environ. Microbiol. 68, 2095-2100 (2002).
35. Lawrence, A. G., Choi, J., Rha, C., Stubbe, J. & Sinskey, A. J. In vitro analysis of the chain termination reaction in the synthesis of poly-(r)-beta-hydroxybutyrate by the class III synthase from Allochromatium vinosum. Biomacromolecules 6, 2113-2119 (2005).
36. Lutz, R. & Bujard, H. Independent and tight regulation of transcriptional units in Escherichia coli via the LacR/O, the TetR/O and AraC/I1-I2 regulatory elements. Nucleic Acids Res. 25, 1203-1210 (1997).
37. Cunningham, F. X. Jr., Sun, Z., Chamovitz, D., Hirschberg, J. & Gantt, E. Molecular structure and enzymatic function of lycopene cyclase from the cyanobacterium Synechococcus sp strain PCC7942. Plant Cell 6, 1107-1121 (1994).
38. Wang, F. & Lee, S. Y. Production of poly(3-hydroxybutyrate) by fed-batch culture of filamentation-suppressed recombinant Escherichia coli. Appl. Environ. Microbiol. 63, 4765-4769 (1997).
39. Pont-Kingdon, G. Creation of chimeric junctions, deletions, and insertions by PCR. in Methods in Molecular Biology: PCR Protocols. vol. 226, edn. 2 (eds. Bartlett, J. M. S. & Strirling, D.) 511-515 (Humana Press Inc., Totowa, N.J., 2003).
40. Tyo, K. E., Zhou, H. & Stephanopoulos, G. N. High-throughput screen for poly-3-hydroxybutyrate in Escherichia coli and Synechocystis sp. strain PCC6803. Appl. Environ. Microbiol. 72, 3412-3417 (2006).

### Example 5

### Subpopulations Balance Model

We developed a subpopulation balance model, based on the models by Bentley et. al¹. These systems of ordinary differential equations will propagate subpopulations, where each subpopulation has a different number of inactive plasmids. Active plasmids are not explicitly accounted for in this model.

This model assumes that the plasmids replicate at the same rate as cells (steady state). For calculations where allele segregation was present, the distribution of plasmids from mother to daughter cell was assumed to be random and was calculated using a binomial distribution as previous¹. The growth rate of each subpopulation will be linearly weighted by the number of inactive plasmids, such that cells with more inactive plasmids will grow faster.

### Methods

Matlab software package was used to integrate the system forward in time.

### Parameters

40 copies of a plasmid
Each plasmid contains a 5 kb region of recombinant expression
Mutation rate—5.4×10−10 errors/by copied2-4
Growth rates (based on measured rates for PHB production)
With 40 active plasmids 0.15 h⁻¹
With 0 active plasmids 0.32 h⁻¹

### Matlab Code

function [N] = seg_model(Npmax,n)
% This population balance model consist of a vector, N, with i indices,
where
% each element has copy number (i−1) of the mutant plasmid. At each
% generation, the distribution of the population is calculated based on the
% growth rate, mutation rate, and probability distribution from mother to
% daughter cell.
% The growth rate is weighted at each generation by a growth advantage
% observed for XL1 Blue growing with

| | |
|---|---|
| % | (a) pZE-tac-pha [Specific growth rate = 0.15 h−1] |
| % | (b) null PHB mutant of pZE-tac-pha [Specific growth rate = |
| | 0.32 h−1] |
| % | |

% The mutation rate is based on literature values for prokaryotes.

| | |
|---|---|
| % | Burger, et.al.Genetics 172, 197-206 (2006). |
| % | Drake, et. al. Genetics 148, 1667-1686 (1998). |
| % | Taft-Benz, et. al. Nucl. Acids Res. 26, 4005-4011 (1998). |
| % | |

% The probability distribution is either

| | |
|---|---|
| % | “Random inheritence” - binomial distribution of mutated |
| % | plasmids from mother to daughter cell, typical of high copy |
| | plasmids |
| % | |
| % | “Ordered inheritence” - each daughter cell receives the exact |
| % | number of mutated plasmids as the mother cell. This would |
| % | be the condition imposed for CIChE constructs. |
| % | |

% The inputs are Npmax, the highest copy number of plasmids, and n, the
% number of generations to calculate.
%%%%%%%%%%%%%%%%%%%%%%%%%%%%%%%%%%%%
% Random inheritance - binomial distribution

| | |
|---|---|
| For | j=1:Npmax+1 |
| | i = 1; |
| | while i−1 <= 2*(j−1) |

deltaij(i,j) = (factorial(2*(j−1))/(factorial((2*(j−1))−(i−1))*

factorial(i− 1)))*0.5\hat{ }(2*(j−1));

i = i + 1;

end

end
% Truncate probabilities for copy numbers above maximum copy number
trunc_prob = sum(deltaij(Npmax+1:2*Npmax+1,:));
deltaij(Npmax+1,:) = trunc_prob;
deltaij(Npmax+2:2*Npmax+1,:) = [ ];
%%%%%%%%%%%%%%%%%%%%%%%%%%%%%%%%%%%%
% Ordered inheritance
% deltaij = eye(Npmax+1);
%%%%%%%%%%%%%%%%%%%%%%%%%%%%%%%%%%%%
% Set growth advantage matrix
slope = (.30 − .15)/(Npmax − 0); % Based on experimental observation
int = .15; % Based on experimental observation
% Make linear growth advantage from slope and intercept given.
y = zeros(Npmax+1,1);

| | |
|---|---|
| for | x=1:Npmax+1 |
| | y(x) = (x−1)*slope + int; |

end

| | |
|---|---|
| for | i=1:Npmax+1 |
| | G(i,i) = y(i); |

end
%%%%%%%%%%%%%%%%%%%%%%%%%%%%%%%%%%%%
% Vector of starting distribution
No = zeros(Npmax+1,1);
% Start with one cell with no inactive plasmids
No(1) = 1;
%%%%%%%%%%%%%%%%%%%%%%%%%%%%%%%%%%%%
% Setup state vector
N = zeros(Npmax+1,1);
%Propogate n generations
disp(‘Number of Generations’);
disp(n);
% Calculate time to grow n generations
t_end = n*log(2)/G(1,1);
options = odeset;
[T,N] = ode15s(@plasmidgrowth, [0,t_end], No, options, deltaij,
G, Npmax);
pts = length(T);
% Convert time into generations
Gens = T/(log(2)/G(1,1));
% Calculate the fraction of active plasmids
Frac_act = zeros(pts,1);

| for | i=1:pts | |
|---|---|---|
| | Active_plas = 0; | |
| | Tot_plas = sum(N(i,:))*(Npmax); | |
| | for | j=1:Npmax+1 |
| | | Active_plas = Active_plas + N(i,j)*(Npmax+1−j); |

End
Frac_act(i) = Active_plas/Tot_plas;

End
plot(Gens,Frac_act);
return
function [dN_dt] = plasmidgrowth(t, N, deltaij, G, Npmax)
% Calculate the weighted distribution of inherited mutated plasmids to the
% next generation
T = deltaij*G*N;
% Calculate the number of spontaneous mutations
Mut_rate = 5.4e−10;
Length_of_one_gene = 5000;

| | |
|---|---|
| for | i=1:Npmax+1 |
| | Length_of_genes(i,i) = Length_of_one_gene*(Npmax−(i−1)); |

end
SG = G/log(2);
M = Mut_rate*Length_of_genes*SG*N;
M = [ 0 M(1:Npmax)‘]’;
% Add the inherited mutations to spontaneous mutations
dN_dt = T + M;
return

### REFERENCE FOR EXAMPLE 5

1. Bentley, W. E. & Quiroga, O. E. Investigation of subpopulation heterogeneity and plasmid stability in recombinant Escherichia coli via a simple segregated model. Biotechnol. Bioeng. 42, 222-234 (1993).
2. Taft-Benz, S. A. & Schaaper, R. M. Mutational analysis of the 3′→5′ proofreading exonuclease of Escherichia coli DNA polymerase III. Nucl. Acids Res. 26, 4005-4011 (1998).
3. Burger, R., Willensdorfer, M. & Nowak, M. A. Why Are Phenotypic Mutation Rates Much Higher Than Genotypic Mutation Rates? Genetics 172, 197-206 (2006).
4. Drake, J. W., Charlesworth, B., Charlesworth, D. & Crow, J. F. Rates of Spontaneous Mutation. Genetics 148, 1667-1686 (1998).

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety for the purposes disclosed above.

We claim:

1. A method for producing a genetically stable tandem gene duplication comprising
integrating into a chromosome of a host cell a nucleic acid construct comprising a nucleic acid sequence that encodes one or more proteins operably linked to one or more promoter sequences, a nucleic acid sequence encoding a selectable marker, and homologous nucleic acid segments flanking the nucleic acid sequences that encode the one or more proteins and the selectable marker, wherein the host cell comprises a functional recombinase,
selecting for tandem gene duplication (TGD) of the nucleic acid sequences that encode the one or more proteins and the selectable marker by culturing the host cell under selective conditions in which the selectable marker confers a growth advantage to the host cell, wherein the TGD is mediated by the functional recombinase, and
stabilizing the TGD by deleting the recombinase or disabling the recombinase.

2. The method of claim 1, wherein increasing the number of copies of the nucleic acid sequence encoding the selectable marker confers increasing growth advantage to the host cell.

3. The method of claim 1, wherein the homologous nucleic acid segments are at least 50% identical, at least 60% identical, at least 70% identical, at least 80% identical, at least 90% identical, or wherein the homologous nucleic acid segments are identical.

4.-9. (canceled)

10. The method of claim 1, wherein the one or more proteins is/are one or more proteins that is/are non-native to the host cell.

11. The method of claim 1, wherein the recombinase is encoded by the host cell.

12. The method of claim 11, wherein the recombinase is encoded by recA.

13. The method of claim 1, wherein the nucleic acid sequence encoding the selectable marker is an antibiotic resistance gene, optionally wherein the antibiotic resistance gene is a chloramphenicol resistance gene or a tetracycline resistance gene, and optionally wherein the selective conditions comprise culturing the cells in medium that contains chloramphenicol or tetracycline.

14.-17. (canceled)

18. The method of claim 1, wherein the nucleic acid sequence encoding the selectable marker is an auxotrophic marker gene, and optionally wherein the selective conditions comprise culturing the cells in a medium that does not supply the metabolite produced by the auxotrophic marker gene.

19. (canceled)

20. The method of claim 1, wherein the step of selecting for TGD comprises successive rounds of culture of the cell under culture conditions that successively require an increase in the number of copies of the nucleic acid sequence encoding the selectable marker.

21. The method of claim 1, wherein the host cell is a bacterial cell, optionally wherein the bacterial cell is an E. coli cell.

22. (canceled)

23. The method of claim 1, wherein the host cell is a eukaryotic cell, optionally wherein the eukaryotic cell is a yeast cell.

24. (canceled)

25. The method of claim 1, wherein there are two homologous nucleic acid segments flanking the nucleic acid sequences that encode the one or more proteins and the selectable marker.

26. (canceled)

27. The method of claim 1, wherein the flanking homologous nucleic acid segments are at least 25, 50, 100, 200, 300, 400, 500, 600, 700, 800, 900, or 1000 nucleotides in length.

28. (canceled)

29. The method of claim 1, wherein the flanking homologous nucleic acid segments are less than: 50%, 40%, 30%, 20%, 10%, 5%, 4%, 3%, 2%, or 1% identical with the genome of the host cell.

30. The method of any of claim 1, wherein the flanking homologous nucleic acid segments are sufficiently non-identical with the genome of the host cell that they cannot recombine with the genome of the host cell in the presence of the functional recombinase, and are sufficiently homologous with each other that they can recombine with each other in the presence of the functional recombinase.

31. The method of claim 1, wherein the flanking homologous nucleic acid segments are derived from a genome other than that of the host cell.

32. The method of claim 1, wherein the host cell is a bacterial cell and the flanking homologous nucleic acid segments are derived from a genome of a different species of bacteria or a eukaryotic cell genome, optionally wherein the host cell is an E. coli cell and the flanking homologous nucleic acid segments are derived from a Synechocystis genome.

33. (canceled)

34. The method of claim 1, wherein the flanking homologous nucleic acid segments are non-coding.

35. The method of claim 1, wherein the nucleic acid sequence that encodes the one or more proteins is inserted in the construct in a multiple cloning site between the flanking homologous nucleic acid segments.

36. The method of claim 1, wherein the cell is not cultured under the selective conditions after the tandem gene duplication is stabilized by deleting the recombinase or disabling the recombinase.

37. The method of claim 1, wherein the nucleic acid sequence that encodes the one or more proteins is a phaECAB, CrtEBI, or dxs-idi-ispDF operon.

38. The method of claim 1, wherein the one or more promoter sequences is one or more promoters that is/are dependent on the native RNA polymerase of the host cell.

39.-41. (canceled)

42. A method for producing a protein or metabolite comprising culturing a cell produced by the method of claim 1 in culture medium.

43. The method of claim 42, further comprising isolating and/or purifying the protein or metabolite from the cell or culture medium.

44. A nucleic acid construct comprising
a nucleic acid sequence that encodes one or more proteins operably linked to one or more promoter sequences functional in a host cell,
a nucleic acid sequence that encodes a selectable marker, and
homologous nucleic acid segments flanking the nucleic acid sequence that encodes the one or more proteins and the nucleic acid sequence that encodes the selectable marker,
wherein the nucleic acid sequence that encodes the one or more proteins is operably linked to the one or more promoter sequences on a multicopy number plasmid vector having an origin of replication during construction of the construct.

45.-48. (canceled)


SEQUENCE LISTING (41 sequences)

SEQ ID NO: 1 - DNA, 24 bp, Artificial Sequence
  Synthetic oligonucleotide
gcaccctacgcatcgccagttctt

SEQ ID NO: 2 - DNA, 26 bp, Artificial Sequence
  Synthetic oligonucleotide
cgctctcgggcaaacttttctgtgtt

SEQ ID NO: 3 - DNA, 52 bp, Artificial Sequence
  Synthetic oligonucleotide
ggaacctcttacgtgccgatcaacggccccgttgtcttcactgatcaacact

SEQ ID NO: 4 - DNA, 25 bp, Artificial Sequence
  Synthetic oligonucleotide
cgttgatcggcacgtaagaggttcc

SEQ ID NO: 5 - DNA, 24 bp, Artificial Sequence
  Synthetic oligonucleotide
ccttaaaaaaattacgccccgccc

SEQ ID NO: 6 - DNA, 36 bp, Artificial Sequence
  Synthetic oligonucleotide
tatcggatccccagttctttcaaaaacgtccacgcc

SEQ ID NO: 7 - DNA, 39 bp, Artificial Sequence
  Synthetic oligonucleotide
gggagctcagcatgcccttaaaaaaattacgccccgccc

SEQ ID NO: 8 - DNA, 40 bp, Artificial Sequence
  Synthetic oligonucleotide
ggaattcaacgcgtccagttctttcaaaaacgtccacgcc

SEQ ID NO: 9 - DNA, 35 bp, Artificial Sequence
  Synthetic oligonucleotide
cgtctagagccccgttgtcttcactgatcaacact

SEQ ID NO: 10 - DNA, 35 bp, Artificial Sequence
  Synthetic oligonucleotide
gcaagcatgcagcttcccaaccttaccagagggcg

SEQ ID NO: 11 - DNA, 30 bp, Artificial Sequence
  Synthetic oligonucleotide
gcacgcgtcggcaggtcagcccatatgcag

SEQ ID NO: 12 - DNA, 20 bp, Artificial Sequence
  Synthetic oligonucleotide
cgcctgatgaatgctcatcc

SEQ ID NO: 13 - DNA, 20 bp, Artificial Sequence
  Synthetic oligonucleotide
aggttttcaccgtaacacgc

SEQ ID NO: 14 - DNA, 20 bp, Artificial Sequence
  Synthetic oligonucleotide
gtgatgccgaaatggttgcc

SEQ ID NO: 15 - DNA, 20 bp, Artificial Sequence
  Synthetic oligonucleotide
gcggtcagacgctgcaactg

SEQ ID NO: 16 - DNA, 34 bp, Artificial Sequence
  Synthetic oligonucleotide
aaagacgtccgttgatcggcacgtaagaggttcc

SEQ ID NO: 17 - DNA, 32 bp, Artificial Sequence
  Synthetic oligonucleotide
aagagctcccttaaaaaaattacgccccgccc

SEQ ID NO: 18 - DNA, 36 bp, Artificial Sequence
  Synthetic oligonucleotide
ggggtaccgcatgactgacgttgtcatcgtatccgc

SEQ ID NO: 19 - DNA, 32 bp, Artificial Sequence
  Synthetic oligonucleotide
cgacgcgtcggaaaaccccttccttatttgcg

SEQ ID NO: 20 - DNA, 36 bp, Artificial Sequence
  Synthetic oligonucleotide
ggggtaccgcatgactcagcgcattgcgtatgtgac

SEQ ID NO: 21 - DNA, 29 bp, Artificial Sequence
  Synthetic oligonucleotide
cgacgcgtccgactggttgaaccaggccg

SEQ ID NO: 22 - DNA, 34 bp, Artificial Sequence
  Synthetic oligonucleotide
ggggtaccgacggcagagagacaatcaaatcatg

SEQ ID NO: 23 - DNA, 30 bp, Artificial Sequence
  Synthetic oligonucleotide
cgacgcgtatggaaacgggagggaacctgc

SEQ ID NO: 24 - DNA, 40 bp, Artificial Sequence
  Synthetic oligonucleotide
gagctgttgacaattaatcatcggctcgtataatgtgtgg

SEQ ID NO: 25 - DNA, 48 bp, Artificial Sequence
  Synthetic oligonucleotide
aattccacacattatacgagccgatgattaattgtcaacagctcacgt

SEQ ID NO: 26 - DNA, 20 bp, Artificial Sequence
  Synthetic oligonucleotide
cgttgtcatcgtatccgccg

SEQ ID NO: 27 - DNA, 20 bp, Artificial Sequence
  Synthetic oligonucleotide
gacttcgctcacctgctccg

SEQ ID NO: 28 - DNA, 20 bp, Artificial Sequence
  Synthetic oligonucleotide
gtggtgttccgcaagatgac

SEQ ID NO: 29 - DNA, 20 bp, Artificial Sequence
  Synthetic oligonucleotide
cgttcaccgacgagatgttg

SEQ ID NO: 30 - DNA, 20 bp, Artificial Sequence
  Synthetic oligonucleotide
ggagcagagccagtatcagg

SEQ ID NO: 31 - DNA, 20 bp, Artificial Sequence
  Synthetic oligonucleotide
caccctggatgtaggagccc

SEQ ID NO: 32 - DNA, 37 bp, Artificial Sequence
  Synthetic oligonucleotide
cggggtaccgccagtcactatggcgtgctgctagcgc

SEQ ID NO: 33 - DNA, 31 bp, Artificial Sequence
  Synthetic oligonucleotide
cgacgcgtggccgcccgcctaaacgggacgc

SEQ ID NO: 34 - DNA, 33 bp, Artificial Sequence
  Synthetic oligonucleotide
cggcatatgagttttgatattgccaaatacccg

SEQ ID NO: 35 - DNA, 35 bp, Artificial Sequence
  Synthetic oligonucleotide
cgcggctagcttatgccagccaggccttgattttg

SEQ ID NO: 36 - DNA, 52 bp, Artificial Sequence
  Synthetic oligonucleotide
cgcggctagcgaaggagatatacatatgcaaacggaacacgtcattttattg

SEQ ID NO: 37 - DNA, 34 bp, Artificial Sequence
  Synthetic oligonucleotide
cgcggaattcgctcacaaccccggcaaatgtcgg

SEQ ID NO: 38 - DNA, 51 bp, Artificial Sequence
  Synthetic oligonucleotide
cggcgaattcgaaggagatatacatatggcaaccactcatttggatgtttg

SEQ ID NO: 39 - DNA, 37 bp, Artificial Sequence
  Synthetic oligonucleotide
gcgctcgagtcattttgttgccttaatgagtagcgcc

SEQ ID NO: 40 - DNA, 33 bp, Artificial Sequence
  Synthetic oligonucleotide
taaaccatgggttttgatattgccaaatacccg

SEQ ID NO: 41 - DNA, 36 bp, Artificial Sequence
  Synthetic oligonucleotide
cggggtacctcattttgttgccttaatgagtagcgc